SEQUENCE LISTING (51 sequences)

SEQ ID NO: 1 - DNA, 821 bp, Homo sapiens
  variation (68)..(68): A or C
aggatcccaaggcccaactccccgaaccactcagggtcctgtggacgctcacctagctgcaatggctncaggctcccggacgtccctgctcctggcttttggcctgctctgcctgncctggcttcaagagggcagtgccttcccaaccattcccttatccaggctttttgacaacgntatgctccgcgcccatcgtctgcaccagctggccnntgacacctacnaggagtttgaagaagcctatatcccaaaggaacagaagtattcattcctgcagancccccagacctccctctgtttctcagagtctattccgacaccctccaacagggaggaaacacaacagaaatccaacctagagctgctccgcatctncctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacnatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccttggaagttgccactccagtgcccaccagccttgtcctaataaaattaagttgcatca

SEQ ID NO: 2 - protein, 26 aa, Homo sapiens
  variation (3)..(3): Thr or Ala
MAXGSRTSLLLAFGLLCLXWLQEGSA

SEQ ID NO: 3 - protein, 191 aa, Homo sapiens
  variation (13)..(13): Ala or Val
FPTIPLSRLFDNXMLRAHRLHQLAXDTYXEFEEAYIPKEQKYSFLQXPQTSLCFSESIPTPSNREETQQKSNLELLRIXLLLIQSWLEPVQFLRSVFANSLVYGASDSNVYDLLKDLEEGIQTLMGRLEDGSPRTGQIFKQTYSKFDTNSHNXDALLKNYGLLYCFRKDMDKVETFLRIVQCRSVEGSCGF

SEQ ID NO: 4 - DNA, 4234 bp, Homo sapiens
  variation (1665)..(1665): A or C
tgccaccacgcccagctaatttttgtacttttagtagagatggagttttgccatgttggctagtctggccttgaactcctgacctcaagtgatccacccacctcaaagccacccaaagtttggggattacaagcgtgagccactgtgtccggcctggagaaaggactttaaatgacgcaatgtaggaagagcaaggttgtggagatctgctgccctggctgaggtagctcatgcaatcagtctctctgagccacagtctcttgatctgtgaaatcggaagaaaataatacctccttcacaagacaagtggcaggtcagatgtgagaagcacagtgcaggccctcggcaactggaaaagctctatacagatctgaaaaggaggaggagaaaaaagaggaggggcttccatggctggacagggcatctttctttttctttttctttttttttttttttttttttttgaggtggagtcttgctctgttgccaaggttggagtgcagcagcacgatctccgctcactgcaagctctgcctcccggattcacgccattctcctgcctcagcctcccgagtagctgggaatacaggcgcccgccactacgcccagctaacttttttgcatttttagtacagagtggatttcacctggttagccaagatggtcttgatctactgacctcgtgatccgcccgcctcggcctcccaaagtgctgggattacaggcatgagccaccgcgcccagcctgatagagcatctttcggcgtgatgtgttctgagttccaaagctgaggaagagactcaaatcttcaagagctcttctaactttgagattctctgatggtttcagggctatgggaggaagagcttgtggtccgtgtctgctcccgggatttctgtttcttggtttgtgtctctgctgcaagtccaaggagctggggcaataccttgagtctgggttcttcgtccccagggacctgggggagccccagcaatgctcagggaaaggggagagcaaagtgtggggttggttctctctagtggtcagtgttggaactgcatccagctgactcaggctgacccaggagtcctcagcagaagtggaattcaggactgaatcgtgctcacaacccccacaatctattggctgtgcttggccccttttcccaacacacacattctgtctggtgggtggaggttaaacatgcggggaggaggaaagggataggatagagaatgggatgtggtcggtagggggtctcaaggactggctatcctgacatccttctccgcgttcaggttggccaccatggcctgcggccagagggcacccacgtgacccttaaagagaggacaagttgggtggtatctctggctgacactctgtgcacaaccctcacaacactggtgacggtgggaagggaaagatgacaagccagggggcatgatcccagcatgtgtgggaggagcttctaaattatccattagcacaagcccgtcagtggccccatgcataaatgtacacagaaacaggtgggggcaacagtgggagagaaggggccagggtataaaaagggcccacaagagaccagctcaaggatcccaaggcccaactccccgaaccactcagggtcctgtggacagctcacctagcggcaatggctncaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggctcccggacgtccctgctcctggcttttggcctgctctgcctgncctggcttcaagagggcagtgccttcccaaccattcccttatccaggctttttgacaacgntatgctccgcgcccatcgtctgcaccagctggccnntgacacctacnaggagtttgtaagctcttggggaatgggtgcgcatcaggggtggcaggaaggggtgactttcccccgctgggaaataagaggaggagactaaggagctcagggtttttcccgaagcgaaaatgcaggcagatgagcacacgctgagtgaggttcccagaaaagtaacaatgggagctggtctccagcgtagaccttggtgggcggtccttctcctaggaagaagcctatatcccaaaggaacagaagtattcattcctgcagancccccagacctccctctgtttctcagagtctattccgacaccctccaacagggaggaaacacaacagaaatccgtgagtggatgccttctccccaggcggggatgggggagacctgtagtcagagcccccgggcagcacagccaatgcccgtccttcccctgcagaacctagagctgctccgcatctncctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacnatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattcgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagaacttacagtctggtaagaggaataaaatgtctgcaaatagccacaggacaggtcaaaggaaggaggggctatttccagctgagggcaccccatcaggaaagcaccccagacttcctacaactactagacacatctcgatgc

SEQ ID NO: 5 - DNA, 17 bp, artificial sequence
  Primer
gggagccccagcaatgc

SEQ ID NO: 6 - DNA, 23 bp, artificial sequence
  primer
acggatttctgttgtgtttcctc

SEQ ID NO: 7 - DNA, 23 bp, artificial sequence
  primer
gagctcagggtttttcccgaagc

SEQ ID NO: 8 - DNA, 23 bp, artificial sequence
  primer
gggcagagataatagcaaacaag

SEQ ID NO: 9 - DNA, 19 bp, artificial sequence
  primer
tgtaggaagtctggggtgc

SEQ ID NO: 10 - DNA, 18 bp, artificial sequence
  primer
tacaggcgtgtgcccaac

SEQ ID NO: 11 - DNA, 18 bp, artificial sequence
  primer
tgccaccacgcccagcta

SEQ ID NO: 12 - DNA, 22 bp, artificial sequence
  primer
atcggaagaaaataatacctcc

SEQ ID NO: 13 - DNA, 20 bp, artificial sequence
  primer
ctgtaatcccagcactttgg

SEQ ID NO: 14 - DNA, 20 bp, artificial sequence
  primer
ctcctcctccttttcagatc

SEQ ID NO: 15 - DNA, 20 bp, artificial sequence
  primer
gatcacgaggtcagtagatc

SEQ ID NO: 16 - DNA, 20 bp, artificial sequence
  primer
ggattcacgccattctcctg

SEQ ID NO: 17 - DNA, 21 bp, artificial sequence
  primer
gtacagagtggatttcacctg

SEQ ID NO: 18 - DNA, 20 bp, artificial sequence
  primer
gtttgtgtctctgctgcaag

SEQ ID NO: 19 - DNA, 18 bp, artificial sequence
  primer
gctgacccaggagtcctc

SEQ ID NO: 20 - DNA, 18 bp, artificial sequence
  primer
ttggccaccatggcctgc

SEQ ID NO: 21 - DNA, 19 bp, artificial sequence
  primer
ccctcacaacactggtgac

SEQ ID NO: 22 - DNA, 19 bp, artificial sequence
  primer
ccccgtcccatctacaggt

SEQ ID NO: 23 - DNA, 19 bp, artificial sequence
  primer
cccctttccctgagcattg

SEQ ID NO: 24 - DNA, 20 bp, artificial sequence
  primer
attgtgggggttgtgagcac

SEQ ID NO: 25 - DNA, 19 bp, artificial sequence
  primer
tgcacagagtgtcagccag

SEQ ID NO: 26 - DNA, 20 bp, artificial sequence
  primer
ttttaggggcgcttacctgt

SEQ ID NO: 27 - DNA, 18 bp, artificial sequence
  primer
cccgtcccatctacaggt

SEQ ID NO: 28 - DNA, 21 bp, artificial sequence
  primer
atttggccaatctcagaaagc

SEQ ID NO: 29 - DNA, 18 bp, artificial sequence
  primer
gctccctctgttgccctc

SEQ ID NO: 30 - DNA, 18 bp, artificial sequence
  primer
ggagctggtctccagcgt

SEQ ID NO: 31 - DNA, 19 bp, artificial sequence
  primer
tatgctccgcgcccatcgt

SEQ ID NO: 32 - DNA, 20 bp, artificial sequence
  primer
atagacgttgctgtcagagg

SEQ ID NO: 33 - DNA, 20 bp, artificial sequence
  primer
ctgcattttcgcttcgggaa

SEQ ID NO: 34 - DNA, 18 bp, artificial sequence
  primer
caggggaaggacgggcat

SEQ ID NO: 35 - DNA, 21 bp, artificial sequence
  primer
gtcggaatagactctgagaaa

SEQ ID NO: 36 - DNA, 20 bp, artificial sequence
  primer
cctccaacagggaggaaaca

SEQ ID NO: 37 - DNA, 18 bp, artificial sequence
  primer
ggcagcacagccaatgcc

SEQ ID NO: 38 - DNA, 21 bp, artificial sequence
  primer
tgagaaagggagggaacagta

SEQ ID NO: 39 - DNA, 20 bp, artificial sequence
  primer
cacacaacgatgacgcacta

SEQ ID NO: 40 - DNA, 20 bp, artificial sequence
  primer
ccaacagggaggaaacacaa

SEQ ID NO: 41 - DNA, 20 bp, artificial sequence
  primer
ctctgacagcaacgtctatg

SEQ ID NO: 42 - DNA, 20 bp, artificial sequence
  primer
tccagcttggttcccaatag

SEQ ID NO: 43 - DNA, 21 bp, artificial sequence
  primer
ctaacacagctctcaaagtca

SEQ ID NO: 44 - DNA, 19 bp, artificial sequence
  primer
cttgccccttgctccatac

SEQ ID NO: 45 - DNA, 20 bp, artificial sequence
  primer
caggttgtcttcccaacttg

SEQ ID NO: 46 - DNA, 20 bp, artificial sequence
  primer
tctaggtcctttaggaggtc

SEQ ID NO: 47 - DNA, 20 bp, artificial sequence
  primer
cgttgtgtgagtttgtgtcg

SEQ ID NO: 48 - DNA, 18 bp, artificial sequence
  primer
gctgacccaggagtcctc

SEQ ID NO: 49 - DNA, 20 bp, artificial sequence
  primer
tcacctagctgcaatggcta

SEQ ID NO: 50 - DNA, 19 bp, artificial sequence
  primer
aaaggccagctggtgcaga

SEQ ID NO: 51 - DNA, 19 bp, artificial sequence
  primer
atggttgggaaggcactgc

(19) United States
(12) Patent Application Publication
(10) Pub. No.: US 2003/0170679 A1
(43) Pub. Date: Sep. 11, 2003

(54) Single nucleotide polymorphisms in GH-1

(76) Inventors: Linda Susan Wood, Portage, MI (US); Susanne Wagner, Salt Lake City, UT (US); Luis A. Parodi, London (GB)

Correspondence Address: PHARMACIA & UPJOHN, 301 HENRIETTA ST, 0228-32-LAW, KALAMAZOO, MI, 49007, US

(21) Appl. No.: 10/289,845

(22) Filed: Nov. 7, 2002

Related U.S. Application Data
(60) Provisional application No. 60/347,448, filed on Nov. 9, 2001.

(51) Int. Cl. C12Q 1/68; C07H 21/04; C12M 1/34

(52) U.S. Cl. 435/6; 435/287.2; 536/23.2

(57) ABSTRACT

The invention provides nucleic acid segments of the GH-1 gene including polymorphic sites. Allele specific primers and probes hybridizing to regions flanking these sites are also provided. The invention also provides methods for diagnosing GH-1 dysfunction.

### CROSS REFERENCE TO RELATED APPLICATIONS

This application claims the benefit of the following provisional application: 60/347,448, filed Nov. 9, 2001, under 35 USC 119(e)(1).

### FIELD OF THE INVENTION

The invention provides nucleic acid segments of a Growth Hormone 1 (GH- 1) gene including polymorphic sites. The invention also provides methods for determining whether an individual suspected of growth hormone dysfunction is a suitable candidate for administration of an agent acting on GH-1 dysfunction.

### BACKGROUND

Single Nucleotide Polymorphisms

All organisms undergo periodic mutation in the course of their evolution and thus generate variant forms of progenitor sequences (Gusella, Ann. Rev. Biochem. 55, 831-854 (1986)). The variant form may or may not confer an evolutionary advantage relative to a progenitor form. The variant form may be neutral. In some instances, a variant form is lethal and is not transmitted to further generations of the organism. In other instances, a variant form confers an evolutionary advantage to the species and is eventually incorporated into the DNA of many or most members of the species and effectively becomes the progenitor form. In many instances, both progenitor and variant form(s) survive and co-exist in a species population. This coexistence of multiple forms of a sequence gives rise to polymorphisms.

Several different types of polymorphism have been reported. A restriction fragment length polymorphism (RFLP) means a variation in DNA sequence that alters the length of a restriction fragment as described in Botstein et al., Am. J. Hum. Genet. 32, 314-331 (1980). The restriction fragment length polymorphism may create or delete a restriction site, thus changing the length of the restriction fragment. RFLPs have been widely used in human and animal genetic analyses (see U.S. Pat. No. 5,856,104, Jan. 5, 1999, Chee, et al, WO 90/13668; WO90/11369; Donis-Keller, Cell 51, 319-337 (1987); Lander et al., Genetics 121, 85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetranucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307, 113-115 (1992); Horn et al., WO 91/14003; Jeffreys, E P 370,719), and in a large number of genetic mapping studies.

Some other polymorphisms take the form of single nucleotide variations between individuals of the same species. Such polymorphisms are far more frequent than RFLPS, STRs and VNTRs. Although it should be recognized that a single nucleotide polymorphism may also result in a RFLP because a single nucleotide change can also result in the creation or destruction of a restriction enzyme site. Some single nucleotide polymorphisms occur in protein-coding sequences, in which case, one of the polymorphic forms may give rise to the expression of a defective or other variant protein and, potentially, a genetic disease. Examples of genes, in which polymorphisms within coding sequences give rise to genetic disease, include beta-globin (sickle cell anemia) and CFTR (cystic fibrosis). Other single nucleotide polymorphisms occur in noncoding regions. Some of these polymorphisms may also result in defective protein expression (e.g., as a result of defective splicing). Other single nucleotide polymorphisms have no phenotypic effects but still may be genetically linked to a phenotypic effect. The greater frequency and uniformity of single nucleotide polymorphisms means that there is a greater probability that such a polymorphism will be found in close proximity to a genetic locus of interest than would be the case for other polymorphisms. Also, the different forms of characterized single nucleotide polymorphisms are often easier to distinguish that other types of polymorphism (e.g., by use of assays employing allele-specific hybridization probes or primers). In a condition such as short stucture in which multiple gene products play a role in the analysis of the disease, SNPs show particular promise as a research tool and they may also be valuable diagnostic tools.

Growth Hormone

Growth hormone 1 (GH-1) is a 191 amino acid globular protein that is released from the anterior pituitary and is vital for normal postnatal growth (Niall H D. Nature 1971;23:90-1; Li CH. Mol Cell Biochem 1982;46:31-41). Insufficient secretion of growth hormone 1 can lead to growth disorders and short stature, affecting from 1 in 4,000 to 1 in 10,000 live births (Phillips III J A and Cogan J D. J Clinical Endocrinology Metabolism 1994;78:11-16.)

While most of the cases are sporadic, three to thirty percent of the individuals have an affected parent or sibling that would suggest a genetic basis for the growth hormone deficiency. There are four forms of familial isolated growth hormone deficiency (IGHD), IGHD IA, IGHD IB, IGHD II and IGHD III (Phillips 1994). Type LA is the most severe form and is autosomal recessively inherited and is caused by homozygous deletions, substitutions or nonsense mutations. The result is an absence of growth hormone that results in severe dwarfism. The most common form is IGHD IB, which is autosomal recessive, is caused by splice site mutations. IGHD II is caused by splice site mutations and is autosomal dominant. IGHD III is X-linked inherited and its cause is unknown. The latter three forms lead to the production of a small amount of growth hormone resulting in dwarfism that usually responds to exogenous growth hormone.

The promoter region of GH-1 has been examined for polymorphisms that would be associated with IGDH (Wagner J K et al. Eur J Endocrinol 1997:137:474-81; Giordano M, Hum Genet 1997;100:249-55. DNA samples were obtained for both short stature individuals and individuals of normal height. Eight (Giordano 1997) and twelve (Wagner 1997) SNPs were identified with seven of the SNPs seen in both studies. Neither study saw any association between the SNPs in the IGHD individuals and the controls. Other GH-1 polymorphisms have been described (WO01/85993)

It is clear that new single nucleotide polymorphisms that are predictive for growth hormone dysfunction meet a pressing need and are the subject of the invention.

### SUMMARY OF THE INVENTION

The invention is based on the discovery of a set of GH-1 gene polymorphic markers. These markers are located in the coding region of GH-1. The sequence of the GH-1 message or cDNA of is set forth below. The polymorphisms with their associated amino acid changes are noted are in bold type.

| aggatcccaaggcccaactccccgaaccactcagggtcctgtggacgctcacctagctgca | |
|---|---|
| | 1↓2 |
| −26 | ATG GCT A/CCA GGC TCC CGG ACG TCC CTG CTC CTG GCT TTT GGC CTG |
| | Met Ala E,UNS Thr Gly Ser Arg Thr Ser Leu Leu Leu Ala Phe Gly Leu |
| | Ala |
| −11 | CTC TGC CTG C/TCC TGG CTT CAA GAG GGC AGT GCC TTC CCA ACC ATT |
| | Leu Cys Leu Pro Trp Leu Gln Glu Gly Ser Ala Phe Pro Thr Ile |
| | Ser |
| 5 | CCC TTA TCC AGG CTT TTT GAC AAC GC/TT ATG CTC CGC GCC CAT CGT |
| | Pro Leu Ser Arg Leu Phe Asp Asn Ala Met Leu Arg Ala His Arg |
| | Val 2↓3 |
| 20 | CTG CAC CAG CTG GCC T/AT/AT GAC ACC TAC C/TAG GAG TTT GAA GAA GCC |
| | Leu His Gln Leu Ala Phe Asp Thr Tyr Gln Glu Phe Glu Glu Ala |
| | Ile Term |
| | Tyr |
| 35 | TAT ATC CCA AAG GAA CAG AAG TAT TCA TTC CTG CAG AA/CC CCC CAG |
| | Tyr Ile Pro Lys Glu Gln Lys Tyr Ser Phe Leu Gln Asn Pro Gln |
| | Thr |
| 50 | ACC TCC CTC TGT TTC TCA GAG TCT ATT CCG ACA CCC TCC AAC AGG |
| | Thr Ser Leu Cys Phe Ser Glu Ser Ile Pro Thr Pro Ser Asn Arg |
| | 3↓4 |
| 65 | GAG GAA ACA CAA CAG AAA TCC AAC CTA GAG CTG CTC CGC ATC TC/GC |
| | Glu Glu Thr Gln Gln Lys Ser Asn Leu Glu Leu Leu Arg Ile Ser |
| | Cys |
| 80 | CTG CTG CTC ATC CAG TCG TGG CTG GAG CCC GTG CAG TTC CTC AGG |
| | Leu Leu Leu Ile Gln Ser Trp Leu Glu Pro Val Gln Phe Leu Arg |
| 95 | AGT GTC TTC GCC AAC AGC CTG GTG TAC GGC GCC TCT GAC AGC AAC |
| | Ser Val Phe Ala Asn Ser Leu Val Tyr Gly Ala Ser Asp Ser Asn |
| 110 | GTC TAT GAC CTC CTA AAG GAC CTA GAG GAA GGC ATC CAA ACG CTG |
| | Val Tyr Asp Leu Leu Lys Asp Leu Glu Glu Gly Ile Gln Thr Leu |
| | 4↓5 |
| 125 | ATG GGG AGG CTG GAA GAT GGC AGC CCC CGG ACT GGG CAG ATC TTC |
| | Met Gly Arg Leu Glu Asp Gly Ser Pro Arg Thr Gly Gln Ile Phe |
| 140 | AAG CAG ACC TAC AGC AAG TTC GAC ACA AAC TCA CAC AAC G/CAT GAC |
| | Lys Gln Thr Tyr Ser Lys Phe Asp Thr Asn Ser His Asn Asp Asp |
| | His |
| 155 | GCA CTA CTC AAG AAC TAC GGG CTG CTC TAC TGC TTC AGG AAG GAC |
| | Ala Leu Leu Lys Asn Tyr Gly Leu Leu Tyr Cys Phe Arg Lys Asp |
| 170 | ATG GAC AAG GTC GAG ACA TTC CTG CGC ATC GTG CAG TGC CGC TCT |
| | Met Asp Lys Val Glu Thr Phe Leu Arg Ile Val Gln Cys Arg Ser |
| 185 | GTG GAG GGC AGC TGT GGC TTC TAG |
| | Val Glu Gly Ser Cys Gly Phe ^{*} |

ctgcccgggtggcatccctgtgacccctccccagtgcctctcctggccttggaagttgccac
tccagtgcccaccagccttgtcctaataaaattaagttgcatca

The sequence set forth above represents the major 22 kDa isoform of GH-1 and represents the coding sequence and the amino acid sequence of the GH-1 polypeptide encoded including the 26 amino acid leader peptide. Lateral numbers refer to amino acid residue numbering. Numbers in bold flanking vertical arrows specify the exon boundaries. The termination codon is marked with an asterisk. The sequence set forth above is found in Genbank as accession number NM_{—}00515 and is designated SEQ ID NO:1 The leader sequence and its encoded amino acids are underlined and in italics. The amino acid sequence of the leader sequence is designated SEQ ID NO:2. It will be appreciated that convention refers to the first amino acid sequence of the leader sequence (Met) to be −26 however in SEQ ID NO:2 this numbering is changed to reflect a positive numbering system with the first Met designated as number 1.

The amino acid sequence of the mature GH-1 polypeptide is set forth above and are also designated SEQ ID NO:4 respectively. The first amino acid of the mature protein is designated by convention to be amino acid number 1. The convention is retained in the numbering of SEQ ID NO:4 with the first amino acid in the mature protein (Phe) being number 1.

It will be appreciated that the RNA and resultant cDNA of the major 22 kDa isoform represented above and in SEQ ID NO:1 is encoded by a genomic sequence with introns. The genomic sequence of the GH-1 gene is set forth in SEQ ID NO:4 and is also delineated in FIG. 1. The genomic reference sequence of SEQ ID NO:4 is derived from Genbank accession number J03071 which was first reported by Chen et al. Genomics 4 479-497 (1989).

The invention comprises the first description of GH-1 diagnostic polynucleotides and their complements comprising GH-1 polymorphic sites designated S1, S2, S3, S4, S5, S6, S7, S8 and S9 suitable for the diagnosis of GH-1 dysfunction or predicting the likelihood of transmitting GH-1 dysfunction to offspring or of use in evaluating therapy. The invention further comprises methods of diagnosis and prediction and administration of agents acting on GH-1 dysfunction.

One embodiment of the invention encompasses isolated polynucleotides consisting of, consisting essentially of, or comprising a contiguous span of nucleotides of SEQ ID NO:1 or 4 and the complements thereof wherein said contiguous span is at least 6, 8, 10, 12, 15, 20, 25, 30, 35, 40, 50, 75, 100, 200, 500, or 800 nucleotides in length and which includes one or more single nucleotide GH-1 polymorphic sites of the invention. The invention also encompasses polynucleotides or probes comprising one or more single nucleotide polymorphisms hybridizing under stringent conditions to a GH-1 gene or transcript.

As an example therefore, the invention therefore provides an isolated polynucleotide consisting of, consisting essentially of, or comprising contiguous nucleotides of at least 10, 12, 15, 20, 25, 30, 35, 40, 50, 75, 100, 200, 500, or 800 nucleotides in length of SEQ ID NO:1 in which the nucleotide position 68 is selected from the group of nucleotides A or C;

SEQ ID NO:4 in which the nucleotide position 1665 is selected from the group of nucleotides A or C;

SEQ ID NO:1 in which the nucleotide position 116 is selected from the group of nucleotides C or T; 5 SEQ ID NO:4 in which the nucleotide position 1973 is selected from the group of nucleotides C or T;

SEQ ID NO:1 in which the nucleotide position 177 is selected from the group of nucleotides C or T;

SEQ ID NO:4 in which the nucleotide position 2034 is selected from the group of nucleotides C or T;

SEQ ID NO:1 in which the nucleotide position 212 is selected from the group of nucleotides T or A;

SEQ ID NO:4 in which the nucleotide position 2069 is selected from the group of nucleotides T or A;

SEQ ID NO:1 in which the nucleotide position 213 is selected from the group of nucleotides T or A;

SEQ ID NO:4 in which the nucleotide position 2070 is selected from the group of nucleotides T or A;

SEQ ID NO:1 in which the nucleotide position 224 is selected from the group of nucleotides C or T;

SEQ ID NO:4 in which the nucleotide position 2081 is selected from the group of nucleotides C or T;

SEQ ID NO:1 in which the nucleotide position 279 is selected from the group of nucleotides A or C;

SEQ ID NO:4 in which the nucleotide position 2345 is selected from the group of nucleotides A or C;

SEQ ID NO:1 in which the nucleotide position 375 is selected from the group of nucleotides C or G;

SEQ ID NO:4 in which the nucleotide position 2533 is selected from the group of nucleotides C or G;

SEQ ID NO:1 in which the nucleotide position 596 is selected from the group of nucleotides G or C;

SEQ ID NO:4 in which the nucleotide position 3007 is selected from the group of nucleotides G or C.

Complements of these segments are also included. The segments can be DNA or RNA, and can be double- or single-stranded. Some segments are 10-20 or 10-50 bases long. Preferred segments are 10-400 bases long.

The invention further provides allele-specific oligonucleotides that hybridize to a GH-1 gene or a transcript derived from that gene or its complement. These oligonucleotides can be probes or primers. SEQ ID NO:4 represents a genomic sequence. SEQ ID NO:1 represents a cDNA or RNA sequence of the major transcript of the GH-1 gene. While a preferred embodiment of the invention encompasses polynucleotide sequences derived from genomic DNA one of ordinary skill recognizes the identity of the nucleotide(s) at polymorphic sites close to intronic sequences may be determined with polynucleotide primers or probes having a different sequence when derived from the sequence of the RNA transcript because of the natural splicing of the mRNA. It will be appreciated that other reference sequences exist including splice variants and the like. To the extent that the GH-1 polymorphisms are present in such altered transcripts the invention encompasses polynucleotides designed to detect the GH-1 polymorphisms in the background of such an alternatively spliced transcript.

The invention further provides a method of classifying a nucleic acid obtainded from an individual. The method determines which nucleotides(s) are present at GH-1 polymorphic sites . Optionally, the bases at each polymorphic are determined simultaneously in one reaction. This type of analysis can be performed on a plurality of individuals who are tested for the presence of a disease phenotype. The presence or absence of disease phenotype or propensity for developing a disease state can then be correlated with a base or set of bases present at the polymorphic sites in the individuals tested.

The present invention therefore further provides a method of diagnosing GH-1 dysfunction or the propensity for transmitting such a phenotype to offspring by determining the presence or absence of a GH-1 haplotype or genotype in a patient by obtaining material from a patient comprising nucleic acid including one or more of the GH1 polymorphic sites. and determining the GH-1 haplotype or genotype.

The invention further provides a method for classifying a GH-1 polypeptide obtained from an individual to determine whether said polypeptide is a GH-1 mutant polypeptide.

The invention also provides a method of evaluating therapy with an agent acting on GH-1 dysfunction for treatment of a patient wherein the identity of a nucleotide occupying at least one GH-1 polymorphic site is determined and evaluating whether the patient should undergo therapy with said agent.

The invention also provides a method of evaluating therapy with an agent acting on GH-1 dysfunction for treatment of a patient comprising determining whether a GH-1 polypeptide obtained from said patient is a GH-1 mutant polypeptide The invention also provides a method of administering human growth hormone comprising administering human growth hormone to a patient previously determined to have a nucleotide at a GH-1 polymorphic site indicating GH-1 dysfunction.

The present invention provides GH-1 mutant polypeptides and nucleic acids encoding them wherein the GH-1 mutant polypeptide is encoded by a GH-1 encoding polymorphic nucleic acid with the polymorphic site encoding the rare allele as shown in Table 1.

The invention further provides primers useful in the amplification of nucleic acid segments comprising the GH6-1 polymorphic sites of the invention.

### BRIEF DESCRIPTION OF THE FIGURES

FIG. 1. Genomic sequence of Growth Hormone 1.

FIG. 1 gives the genomic sequence for human growth hormone 1 derived from the Genbank database entry J03071. The polymorphic sites are underlined in bold italic type. The primers used in Example 1 to generate the PCR fragments and to sequence the fragments are underlined and the name of the oligonucleotide and its orientation is indicated above the sequence. The amino acid sequence is below the nucleotide sequence. The first 26 amino acids (−26 to −1) represent a signal sequence peptide. There are 4 introns within the coding region. An arrow indicates the beginning and the end of the gene. The initiation methione, stop codon and poly A addition site are in bold type. The TATA box at −30 to −25 and the two PIT-1 sites at -132 to 107, and −92 to −67 are boxed.

### BRIEF DESCRIPTION OF THE SEQUENCE LISTING

SEQ ID NO:1 GH-1 cDNA sequence with polymorphic sites noted

SEQ ID NO:2 GH-1 signal polypeptide peptide sequence

SEQ ID NO:3 GH-1 mature polypeptide sequence

SEQ ID NO:4 GH-1 Genomic Sequence

SEQ ID NO:5-51 Primers

### DETAILED DESCRIPTION OF THE INVENTION

Definitions

The term “GH-1 diagnostic polynucleotide” means any polynucleotide derived from a GH-1 genomic sequence or a transcript derived from the GH-1 gene comprising a GH-1 polymorphic site (including complements) the forms of major and alternate transcript species are well known in the art. The message sequence of the major isoform is given in SEQ ID NO:1 and the corresponding genomic sequence in SEQ ID NO:4. A diagnostic polynucleotide may be a primer or probe.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064. However, the polynucleotides of the invention are preferably comprised of greater than 50% conventional deoxyribose nucleotides, and most preferably greater than 90% conventional deoxyribose nucleotides The polynucleotide sequences of the invention may be prepared by any known method, including synthetic, recombinant, ex vivo generation, or a combination thereof, as well as utilizing any purification methods known in the art.

The term “isolated” is used herein to describe a polynucleotide or polynucleotide vector of the invention which has been separated to some extent from other compounds with which it is naturally and necessarily usually associated including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially isolated when at least about 50%, preferably 60 to 75% of a sample exhibits a single polynucleotide sequence and conformation (linear versus covalently close). A substantially isolated polynucleotide typically comprises about 50%, preferably 60 to 90% weight/weight of a nucleic acid sample, more usually about 95%, and preferably is over about 99% pure. Polynucleotide purity or homogeneity may be indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The term “purified” when referring to a polypeptide of the invention means separated from the original cellular or organismic environment in which the polypeptide or is normally found. Optionally such a purified polypeptide may be reconstituted with a pharmaceutically acceptable carrier for administration to a patient.

The term primer refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “probe” or “hybridization probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified by hybridization. “Probes” or “hybridization probes” are nucleic acids capable of binding in a base-specific manner to a complementary strand of nucleic acid. Such probes include peptide nucleic acids, as described in Nielsen et al., Science 254, 1497-1500 (1991).

Hybridizations are usually performed under “stringent conditions”, for example, at a salt concentration of no more than 1 M and a temperature of at least 25° C. For example, conditions of 5X SSPE (750 mM NaCl, 50 mM NaPhosphate, 5 mM EDTA, pH 7.4) and a temperature of 25°-60° C. are suitable for allele-specific probe hybridizations. Although this particular buffer composition is offered as an example, one skilled in the art, could easily substitute other compositions of equal suitability.

The term “sequencing,” as used herein, means a process for determining the order of nucleotides in a nucleic acid. A variety of methods for sequencing nucleic acids are well known in the art. Such sequencing methods include the Sanger method of dideoxy-mediated chain termination as described, for example, in Sanger et al., Proc. Natl. Acad. Sci. 74:5463 (1977), which is incorporated herein by reference (see, also, “DNA Sequencing” in Sambrook et al. (eds.), Molecular Cloning: A Laboratory Manual (Second Edition), Plainview, N.Y.: Cold Spring Harbor Laboratory Press (1989), which is incorporated herein by reference). A variety of polymerases including the Klenow fragment of E. coli DNA polymerase I; Sequenase TM (T7 DNA polymerase); Taq DNA polymerase and Amplitaq can be used in enzymatic sequencing methods. Well known sequencing methods also include Maxam-Gilbert chemical degradation of DNA (see Maxam and Gilbert, Methods Enzymol. 65:499 (1980), which is incorporated herein by reference, and “DNA Sequencing” in Sambrook et al., supra, 1989). Once skilled in the art recognizes that sequencing is now often performed with the aid of automated methods.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to GH-1 dysfunction; or to refer to an individual's response to an agent acting on GH-1 dysfunction; or to refer to symptoms of, or susceptibility to side effects to an agent acting on GH-1 dysfunction.

The term “individual suspected of GH dysfunction” means an individual exhibiting one or more of the following characteristics. (i) growth failure, defined as a growth pattern [delineated by a series of height measurements; Brook CDG (Ed) Clinical Pediatric Endocrinology 3rd Ed, Chapter 9, p141 (1995, Blackwell Science)] which, when plotted on a standard height chart [Tanner et al Arch Dis Child 45 755-762 (1970)], predicts an adult height for the individual which is outside the individual's estimated target adult height range, the estimate being based upon the heights of the individual's parents. The present invention therefore further provides a variant of GH1 detected by or detectable according to the above-described method of this invention. Useful as a reference for criterion (i) is Tanner and Whitehouse Arch Dis Child 51 170-179 (1976)]. A patient's target adult height range is calculated as the mid-parental height (MPH) with the range being the 10th to 90th centile for MPH, which is sex-dependent: MPH if male=[father's height+(mother's height+13)]/2 + or − in the range of from 6 to 8 cm, usually 7.5 cm; and MPH if female=[(father's height−13)+mother's height]/2 + or − in the range of from 6 to 8 cm, usually 6 cm; (ii) height velocity below the 25^{th} centile for age; and/or (iii) bone age delay according to the Tanner-Whitehouse scale of at least two years, when compared with chronological age; and/or With respect to the criteria (ii) and (iii), each criterion may be assessed according to known methods and parameters readily available and described in the art, as elaborated further below: (ii) Tanner J M, Whitehouse R H Atlas of Children's Growth (1982, London: Academic Press); and Butler et al Ann Hum Biol 17 177-198 (1990) are sources for statistics enabling a determination of the first criterion, viz that the height velocity of the patient is less than the 25^{th} centile for the patient's age. (iii) The Tanner-Whitehouse scale for assessing years of bone age delay is described by Tanner J M, Whitehouse R H, Cameron N et al in Assessment of Skeletal Maturity and Prediction of Adult Height (1983, London: Academic Press). In the method of this invention, the individual preferably exhibits bone age delay of about 3.5 to 4 years (when compared with chronological age).

Assessment of bone age delay in an individual is subject to a greater level of variation, when carried out more than once, the younger the individual, so, for example, multiple assessments of a child of age two may result in a bone age delay varying by +/−6 months, but at age 3 might vary by +/−4 months, and so on.

Optionally, the patient may also have been subjected to one or more growth hormone function tests. The term “growth hormone function tests” refers to tests of growth hormone secretion, such as those stimulation tests mentioned hereinbefore, particularly the insulin-induced hypoglycemic test (IST). GH function tests are usually carried out on patients who are short; have been clinically assessed and had their height monitored over more than one visit to the endocrine clinic; have no other detectable cause for their growth failure; and therefore warrant being subjected to an assessment of their ability to produce growth hormone secretion from their pituitary gland following an appropriate stimulus, such as the profound drop in blood glucose that results from the administration of intravenous insulin. Often the results of the individual's growth hormone function tests are normal.

It should be noted that the above description refers to children however adults may also be “an individual suspected of GH-1 dysfunction. There is evidence that growth hormone deficiency in adults is deleterious, increasing the risk of death from cardiovascular disease. As compared with age- and sex-matched normal subjects, adults with growth hormone deficiency have increased fat mass, reduced muscle mass and strength, smaller hearts and lower cardiac output, lower bone density, and higher serum lipid concentrations. They also have decreased vitality, energy, and physical mobility; emotional liability; feelings of social isolation; and disturbances in sexual function, despite adequate correction of hormonal deficiencies other than growth hormone deficiency. Vance and Mauras (1999) New England Journal of Medicine 341(16) pp 1206-1216.

The term “GH-1 dysfunction” means a clinical condition including short stature caused by a failure of endogenous GH-1 polypeptide to be produced at normal levels, or to be maintained at normal levels, or to function normally if present at normal levels. A single GH-1 polypeptide when functioning normally at a cellular level binds two GH receptor molecules (GHR) causing them to dimerise. Dimerisation of the two GH-1 bound GHR molecules is believed to be necessary for signal transduction, which is associated with the tyrosine kinase JAK-2. It has been suggested that the diverse effects of GH-1 may be mediated by a single type of GHR molecule that can possess different cytoplasmic domains or phosphorylation sites in different tissues. When activated by JAK-2, these differing cytoplasmic domains can lead to distinct phosphorylation pathways, one for growth effects and others for various metabolic effects. The clinical manifestations of“GH-1 dysfunction” are outlined above.

An “agent acting on GH-1 dysfunction” includes any drug or compound known in the art that addresses, reduces or alleviates one or more symptoms of GH-1 dysfunction. “Agents acting on a GH-1 dysfunction” includes any drug or a compound modulating the activity or concentration of an hormone or regulatory molecule involved in a GH-1 dysfunction that is known in the art. Exogenous growth hormone either recombinantly or naturally produced is encompassed by this definition.

The term “genotype” as used herein refers the identity of the alleles present in an individual or a sample. In the context of the present invention a genotype preferably refers to the description of the polymorphic alleles present in an individual or a sample. The term “genotyping” a sample or an individual for a polymorphic marker consists of determining the specific allele or the specific nucleotide carried by an individual at a polymorphic marker.

The term “haplotype” refers to the actual combination of alleles on one chromosome. In the context of the present invention a haplotype preferably refers to a combination of polymorphisms found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. Polymorphism refers to the occurrence of two or more genetically determined alternative sequences or alleles in a population. Preferred polymorphisms have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A biallelic polymorphism has two forms. A triallelic polymorphism has three forms.

A “single nucleotide polymorphism” (SNP) is a single base pair change. A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. It should be noted that a single nucleotide change could result in the destruction or creation of a restriction site. Therefore it is possible that a single nucleotide polymorphism might also present itself as a restriction fragment length polymorphism. Single nucleotide polymorphisms (SNPs) can be used in the same manner as RFLPs, and VNTRs but offer several advantages. Single nucleotide polymorphisms occur with greater frequency and are spaced more uniformly throughout the genome than other forms of polymorphism. (SNPs) occur at a frequency of roughly 1/1000 base pairs, and are distinguished from rare variations or mutations by a requirement for the least abundant allele to have a frequency of 1% or more (Brookes, 1999). Examples of SNP include:

1. Non-synonymous coding region changes which substitute one amino acid for another in the protein product encoded by the gene,

2. Synonymous changes which do alter amino acid coding sequence due to degeneracy of the genetic code,

3. Changes in promoter, enhancer or other genetic control element sequence which may or may not alter transcription of the gene,

4. Changes in untranslated regions of the mRNA, particularly at the 5'end which may alter the efficiency of ribosomal binding, initiation or translation, or at the 3'end which may alter mRNA stability, and

5. Changes within intronic regions, which may alter the splicing of the transcript or the function of other genetic regulatory elements.

The term “GH-1 polymorphism” is used herein to mean -a polymorphism or polymorphic site disclosed herein within the gene for GH-1. A GH-1 single nucleotide polymorphism is a polymorphism, which reflects variation at a single nucleotide. The term “at least one polymorphism within GH-1” means at least one polymorphism within the GH-1 gene. It is appreciated that the same GH-1 polymorphism potentially exists in all the various transcripts of the GH-1 gene and that the appropriate flanking sequence can be deduced by simple comparison of the relevant sequences.

The term “GH-1 polymorphic site” is used herein to mean a site at which a polymorphism herein described resides. The sites disclosed herein are delineated in Table 1 below and are designated for convenience as S1, S2, S3, S4, S5, S6, S7, S8 and S9 and designated S1, S2, S3, S4, S5, S6, S7, S8 and S9 which are exemplified by the nucleotides at position, 68, 116, 177, 212, 213, 224, 279, 375 or 596 of SEQ ID NO:1 or positions 1665, 1973, 2034, 2069, 2070, 2081, 2345, 2533 or 3007 of SEQ ID NO:4 respectively. It is appreciated that the same GH-1 polymorphic site exists in all the various transcripts of the GH-1 gene and that the appropriate flanking sequence of a GH-1 polymorphic site can be deduced by simple comparison of the relevant sequences.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from 3′ the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The location of nucleotides in a polynucleotide with respect to the end of the polynucleotide are described herein in the following manner. A nucleotide is “at the end” of a polynucleotide if it is at either the 5′ or 3′ end of the polynucleotide.

The term “upstream” is used herein to refer to a location, which, is toward the 5′ end of the polynucleotide from a specific reference point. The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4^{th} edition, 1995).

The terms “complementary” or “complement thereof are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “GH-1 mutant polypeptide” is used herein to mean a GH-1 polypeptide encoded by GH-1 gene or transcript or a portion thereof which comprises at least one GH-1 polymorphic site with the polymorphic site encoding the rare allele as shown in Table 1. Therefore the term GH-1 mutant polypeptide encompasses a polypeptide species comprising SEQ ID NO:3 wherein one or more of positions 13, 25, 29, 47, 79 or 153 is occupied by the amino acid coded for by the rare allele. (i.e. position 13=Val, position 25=Ile or Tyr, position 47=Thr, position 79=Cys, and/or position 153=His or conservative substitutions at these positions). It will be appreciated that the numbering system here makes reference to the numbering relative to the most abundant isoform of the GH-1 protein. The definition is intended to encompass mutations within the framework of other isoforms well known in the art. When reference is made for example, to “a GH-1 mutant polypeptide wherein the amino acid at position 13 is valine” it is intended to that the phrase encompass GH-1 mutant polypeptides derived from other isoforms having the same substitution.

A conservative substitution is recognized in the art as a substitution of one amino acid for another amino acid that has similar properties. Exemplary conservative substitutions are set out in Table A (from WO 97/09433, page 10, published Mar. 13, 1997 (PCT/GB96/02197, filed Sep. 6, 1996), immediately below.

Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. NY:N.Y. (1975), pp.71-77] as set out immediately below.

Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Further examples of grouping of conservative substitutions are set out below.

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Polymorphisms of the Invention

Growth hormone 1 (GH-1) is a 191 amino acid globular protein that is released from the anterior pituitary and is vital for normal postnatal growth (Niall 1971; Li 1982). The pre-hGH-1 has an amino-terminal 26 amino acid signal sequence that directs the protein out of the rough endoplasmic reticulum. The gene for growth hormone 1 (GH-1 gene) is one of five genes found in a cluster spanning 48 kb on chromosome 17 (George 1981). The other four genes are growth hormone 2 (GH-2 gene), chorionic somatomammotropin 1 and 2 (CSH-1 and CSH-2 genes), and a CSH pseudogene (CSHP-1 psuedogene). Each gene has the same exon-intron structure and the five genes are 91-95% similar to each other. Despite their similarities these genes do show tissue-specific expression where GH-1 is transcribed only in the anterior pituitary while the other four genes are transcribed in the placenta (Chen 1989). This tissue-specific transcription is mediated by two binding sites in the promoter region of GH-1 for the pituitary-specific transcriptional factor Pit-1/GHF1 (Bodner 1988). The four placental genes have in their promoter region pituitary-specific repressor sequences (Nachtigal 1992).

The nucleotide and amino acid sequence of the GH-1 cDNA has been disclosed previously in Genbank accession number NM_{—}00515 and is included here as SEQ ID NO:1.

The genomic sequence for the entire growth hormone locus has been reported in Chen et. al. Genomics 4 479-497 (1989) and is in Genbank as accession number J03071.

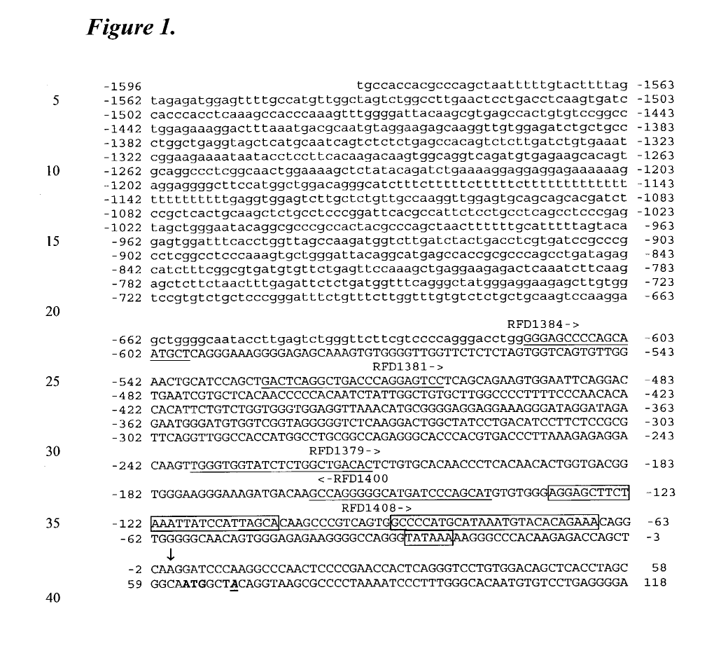

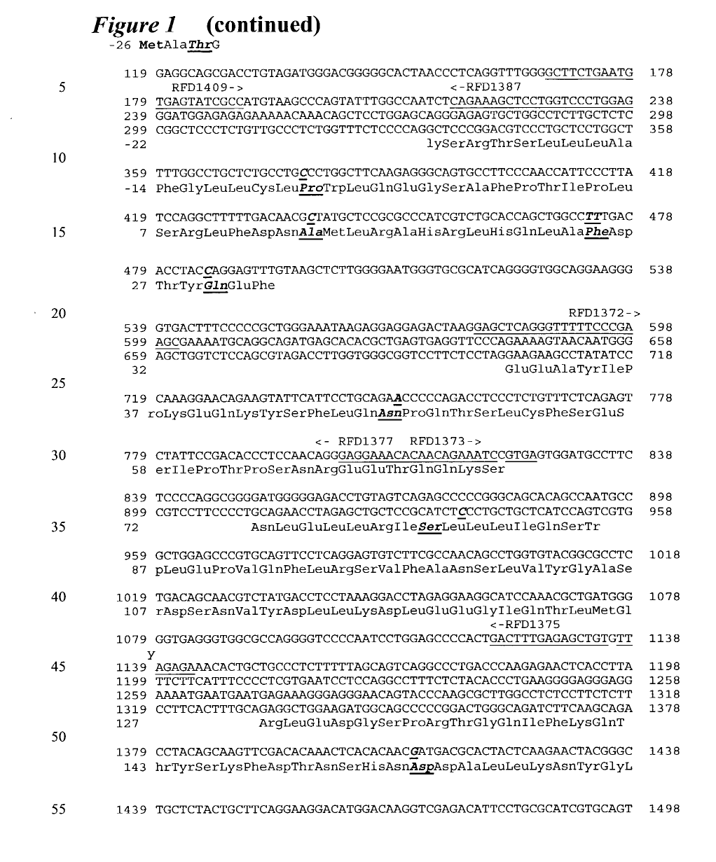

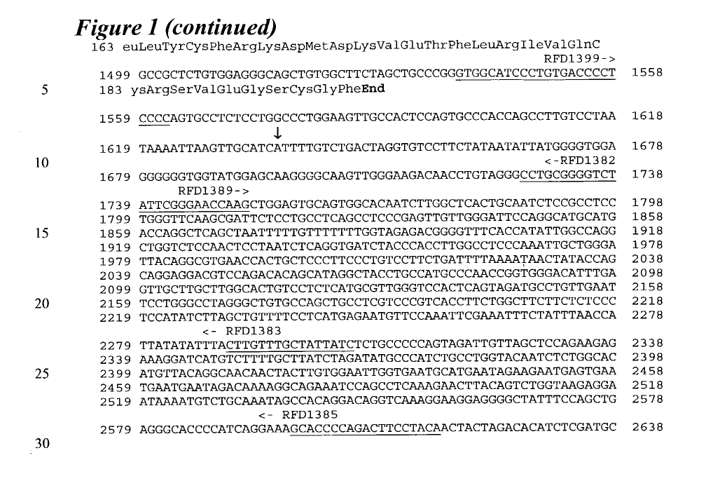

Several different GH isoforms are generated from expression of the GH-1 gene (The GH-1 genomic reference sequence is shown in FIG. 1 and SEQ ID NO:4). In 9% of GH-1 transcripts, exon 2 is spliced to an alternative acceptor splice site 45 bp into exon 3, thereby deleting amino acid residues 32 to 46 and generating a 20 kDa isoform instead of the normal 22 kDa protein. This 20 kDa isoform appears to be capable of stimulating growth and differentiation. The factors involved in determining alternative acceptor splice site selection are not yet characterized but are clearly of a complex nature. A 17.5 kDa isoform, resulting from the absence of codons 32 to 71 encoded by exon 3, has also been detected in trace amounts in pituitary tumor tissue. Splicing products lacking either exons 3 and 4 or exons 2, 3 and 4 have been reported in pituitary tissue but these appear to encode inactive protein products. A 24 kDa glycosylated variant of GH has also been described. The amino acid sequence of the major 22 kDa isoform is presented in SEQ ID NO:3.

The gene encoding GH-1 is located on chromosome 17q23 within a cluster of five related genes. This 66.5 kb cluster has now been sequenced in its entirety [Chen et al. Genomics 4 479-497 (1989). The other loci present in the growth hormone gene cluster are two chorionic somatomammotropin genes (CSH1 and CSH2), a chorionic somatomammotropin pseudogene (CSHP1) and a growth hormone gene (GH2). These genes are separated by intergenic regions of 6 to 13 kb in length, lie in the same transcriptional orientation, are placentally expressed and are under the control of a downstream tissue-specific enhancer. The GH-2 locus encodes a protein that differs from the GH1-derived growth hormone at 13 amino acid residues. All five genes share a very similar structure with five exons interrupted at identical positions by short introns, 260 bp, 209 bp, 92 bp and 253 bp in length in the case of GH-1.

Exon 1 of the GH-1 gene contains 60 bp of 5′ untranslated sequence (although an alternative transcriptional initiation site is present at −54), codons −26 to −24 and the first nucleotide of codon −23 corresponding to the start of the 26 amino acid leader sequence. Exon 2 encodes the rest of the leader peptide and the first 31 amino acids of mature GH. Exons 3-5 encode amino acids 32-71, 72-126 and 127-191, respectively. Exon 5 also encodes 112 bp 3′ untranslated sequence culminating in the polyadenylation site. An Alu repetitive sequence element is present 100 bp 3′ to the GH1 polyadenylation site. Although the five related genes are highly homologous throughout their 5′ flanking and coding regions, they diverge in their 3′ flanking regions.

The GH-1 and GH-2 genes differ with respect to their mRNA splicing patterns. As noted above, in 9% of GH1 transcripts, exon 2 is spliced to an alternative acceptor splice site 45 bp into exon 3 to generate a 20 kDa isoform instead of the normal 22 kDa. The GH-2 gene is not alternatively spliced in this fashion. A third 17.5 kDa variant, which lacks the 40 amino acids encoded by exon 3 of GH1, has also been reported.

The CSH1 and CSH2 loci encode proteins of identical sequence and are 93% homologous to the GH1 sequence at the DNA level. By comparison with the CSH gene sequences, the CSHP1 pseudogene contains 25 nucleotide substitutions within its “exons” plus a G→A transition in the obligate +1 position of the donor splice site of intron 2 that partially inactivates its expression.

By judicious selection of sequencing and PCR primers we have obtained sequence specifically from the GH-1 gene and have identified several heretofore-unknown single nucleotide polymorphisms (outlined in Table 1 below) the presence of which is diagnostic for GH-1 dysfunction or which have utility as genetic markers with a unique position within the human genome.

TABLE 1

| | Mutation Position | | SEQ ID NO: 4 | Common/Rare | Resultant Amino |
|---|---|---|---|---|---|
| | | SEQ ID NO: 1 | Genomic sequence | Allele | Acid Change |
| S1 | 69 | 68 | 1665 | A/C | Thr-24/Ala |
| S2 | 377 | 116 | 1973 | C/T | Pro-8/Ser |
| S3 | 438 | 177 | 2034 | C/T | Ala13/Val |
| S4 | 473 | 212 | 2069 | T/A | Phe25/Ile |
| S5 | 474 | 213 | 2070 | T/A | Phe25/Tyr |
| S6 | 485 | 224 | 2081 | C/T | Gln29/Ter |
| S7 | 749 | 279 | 2345 | A/C | Asn47/Thr |
| S8 | 937 | 375 | 2533 | C/G | Ser79/Cys |
| S9 | 1411 | 596 | 3007 | G/C | Asp153/His |

As noted above, the GH-1 single nucleotide polymorphism at position 68 of the cDNA sequence of SEQ ID NO:1 corresponds to the same polymorphism at position 1665 of the genomic sequence of SEQ ID NO:4. The same concurrence is true of the other polymorphisms of the invention. A similar concurrence could be determined from any other message transcript derived from a GH-1 genomic sequence. It will therefore be appreciated that other reference sequences whether they are derived from splice variants of the GH-1 gene transcript or whether they contain other nucleotide changes would still have an equivalent polymorphic site and that polynucleotides derived from such sequences would be a part of the invention (and are herein defined as GH-1 diagnostic polynucleotides).

There are two distinct types of analysis depending whether a polymorphism in question has already been characterized. The first type of analysis is sometimes referred to as de novo identification. The second type of analysis is determining which form(s) of an identified polymorphism are present in individuals under test. The first type of analysis compares target sequences in different individuals to identify points of variation, i.e., polymorphic sites. By analyzing a groups of individuals representing the greatest ethnic diversity among humans and greatest breed and species variety in plants and animals, patterns characteristic of the most common alleles/haplotypes of the locus can be identified, and the frequencies of such populations in the population determined. Additional allelic frequencies can be determined for subpopulations characterized by criteria such as geography, race, or gender. An example describing the de-novo identification of the polymorphisms of the invention is described below.

### Example 1

### De-Novo Identification of Polymorphisms of the Invention

### Materials and Methods

DNA Samples

DNA samples were obtained from anonymous blood samples. DNA was prepared using the QiaAmp DNA blood mini kit (Qiagen). The samples are referred to as the Population Control Western Michigan samples and labeled CON01 and represent primarily Caucasian and black individuals of varied ethnicity with essentially no with only general phenotypic information known for each individual. (At least one individual was of short stature).

PCR Amplification of GH-1

Primer sequences were designed to be unique to the GH-1 gene and to have at least two nucleotide mismatches with any other related gene in the GH cluster. PCR was performed using Expand High Fidelity enzyme mix in a roughly 50 μl reaction according to the manufacturer's instructions, using a ABI 9600 thermocycler.

The cycling program was as follows: 1 cycle of 94° C. for 2 min then 10 cycles at 94° C. for 15 sec, then 68° C. for 2 min decreasing 1° C. each cycle and then 50 cycles of 94° C. 15 sec, 58° C. 30 sec, 72° C. 2 min.

The reaction mix was composed as follows: 36 μl H₂O, 5 μl 10 TT buffer (140 mM Ammonium Sulfate, 0.1% gelatin, 0.6 M Tris-tricine pH 8.4), 5 μl 15 mM MgSO4, 2 μl 10 mM dNTPs, 1 μl (100 ng, 50 ng or 25 ng) of human genomic DNA (Clontech), 0.4 ,μl Expand High Fidelity enzyme mix (3.5 U/μl)(Roche).

A) 0.3 μl of RFD1384 (1 μg/μl),
0.3 μl of RFD1377 (1 μg/μl),
B) 0.3 μl of RFD1372 (1 μg/μl),
0.3 μl of RFD1383 (1 μg/μl),
C) 0.3 μl of RFD1372 (1 μg/μl),
0.3 μl of RFD1385 (1 μg/μl),

| | | |
|---|---|---|
| RFD1384: | GGGAGCCCCAGCAATGC | (SEQ ID NO:5) |
| RFD1377: | ACGGATTTCTGTTGTGTTTCCTC | (SEQ ID NO:6) |
| RFD1372: | GAGCTCAGGGTTTTTCCCGAAGC | (SEQ ID NO:7) |
| RFD1383: | GGGCAGAGATAATAGCAAACAAG | (SEQ ID NO:8) |
| RFD1385: | TGTAGGAAGTCTGGGGTGC | (SEQ ID NO:9) |

The PCR products were purified using MultiScreen-PCR Filter Plates (Millipore). The PCR reaction was loaded onto the plate and the plate was placed on top of the MultiScreen manifold (Millipore) and a vacuum of 24 inches Hg was applied for 5-10 minutes. The plate was removed from the manifold and 50 μl of H₂O was added to each well. The plate was placed on a plate mixer and shook vigorously for 5 minutes. The purified PCR product was recovered from each well and placed into a new 96 well reaction plate.

DNA Sequencing

The PCR fragments were sequenced directly using an ABI377 fluorescence-based sequencer (Perkin Elmer/Applied Biosystems Division, PE/ABD, Foster City, Calif.) and the ABI BigDye™ Terminator Cycle Sequencing Ready Reaction kit with Taq FSTM polymerase. Each cycle-sequencing reaction contained 9.6 μl of H₂O, 8.4 μl of BigDye Terminator mix (8 μl of Big Dye Terminator and 0.4 μl of DMSO), 1 μl DNA (˜0.5 μg), and 1 μl primer (25 ng/μl) and was performed in a Perkin-Elmer 9600. Cycle-sequencing was performed using an initial denaturation at 98° C. for 1 min, followed by 50 cycles: 96° C. for 30 sec, annealing at 50° C. for 30 sec, and extension at 60° C. for 4 min Extension products were purified using AGTC® gel filtration block (Edge BiosSystems, Gaithersburg, Md.). Each reaction product was loaded by pipette onto the column, which was then centrifuged in a swinging bucket centrifuge (Sorvall model RT6000B tabletop centrifuge) at 750×g for 2 min at room temperature. Column-purified samples were dried under vacuum for about 60 min and then dissolved in 2 μl of a DNA loading solution (83% deionized formamide, 8.3 mM EDTA, and 1.6 mg/ml Blue Dextran). The samples were then heated to 90° C. for 2.3 min and 0.75 μl of each sample was loaded into the gel sample wells for sequence analysis by the ABI377 sequencer. The sequence chromatograms were analyzed using the computer program phred/Phrap and Consed.

Results

FIG. 1 gives the genomic sequence for human growth hormone 1 derived from Genbank J03071. The gene contains four introns within the coding region. To amplify only the gene for growth hormone 1 primers were designed from areas of the gene that are the most dissimilar than the other four genes in the cluster. Several combinations were tried but the most consistent results were obtained by dividing the sequence into two overlapping fragments that span 2.8 kb sequence. This region includes 600 bp of 5′ flanking sequence, all five exons and four introns and 1 kb of 3′ flanking sequence. FIG. 2 shows fragment RFD1984 to RFD1377 (1.5 kb), RFD1372 to RFD1383 (1.8 kb), and RFD1372 to RFD1385 (2.1 kb) with 25 ng, 50 ng or 100 ng of genomic DNA. RFD1384-1377 and RFD1372-1383 give a strong band with all 3 concentrations. RFD1372-1385 does not give a band with 25 ng DNA, a weak band with 50 ng and a fairly strong band with 100 ng.

A plate containing the DNA from 72 individuals, referred to as the Population Control Western Michigan samples (labeled CON01), was amplified using primers for the 1.5 kb and 1.8 kb fragments of growth hormone 1. The PCR products were purified and sequenced. The chromatograms were analyzed with the computer program POLYPHRED, which compares the sequence of the 72 individuals and indicates differences in the sequence. While this sample size is small it has been calculated that for a rare allele with a frequency greater than five percent, it is necessary to compare 48 haploid genomes to detect 99% of the SNPs (Kruglyak 2001). To identify 99.9% of the SNPs with a frequency of one percent would take 192 haploid genomes and our study has 144 haploid genomes so we should detect 97% of the SNPs.

Two of the novel SNPs we found are in the coding region and result in an amino acid change and are outlined below.

TABLE 2

| Position | DNA | | Common | Rare | | Percent rare |
|---|---|---|---|---|---|---|
| | Region | Effect | Heterozygotes | Heterozygotes | Heterozygotes | allele |
| 69 | Exon 1 | Thr→Ala | AA = 71 | GG = 0 | AG = 1 | 0.7 |
| 1411 | Exon 5 | Asp→His | GG = 71 | CC = 0 | GC = 1 | 0.7 |

The SNP in exon 5 changes an aspartic acid to a histidine, which is a change from an acidic amino acid to a weak basic amino acid. It is possible that this change could have an affect on GH-1 in the same way that the Asp¹⁷¹ to His¹⁷¹ change has for species specificity (Souza 1995).

A similar approach using a more diverse sampling of donor samples (including short stature individuals is described in Example 2 below

### Example 2

### Identification of Polymorphisms in Affected and Non-Affected Populations

### Sample Selection Preparation

DNA samples were obtained from the following populations:

Michigan: 219 blood samples from clinical trials volunteers from Michigan. Disease-free, normal height distribution, mostly Caucasian.

GCI: 182 individuals with heights in the lower 2.5% of the population. No confounding conditions.

CRV: 93 individuals from 5 ethnic groups (Caucasian, African-American, Japanese, Chinese, SE Asian and Amerindian) from Coriell

Samples were prepared roughly as described in Example 1

Primer Design

Genomic sequence for the five GH homologues was retrieved from public databases and aligned to each other. The alignment identified areas of highest and lowest conservation between the five genes. Primers were deliberately positioned to contain as much sequence specificity for GHI as possible. In particular, primary primers (labeled a and p) were selected from areas unique to GHI wherever possible.

Nested PCR

Each amplicon was obtained by nested PCR. Two rounds of PCR with primers containing bases unique for GHI increases the specificity of the final product.

Each amplicon was PCR amplified from DNA from eight random population samples and sequenced. The sequence traces of those eight samples were analyzed for the presence of heterozygous positions that appear in every sample, an indication that multiple genes with single base differences have been amplified during PCR. None of the amplicons contained a heterozygous position in all samples.

In addition, several positions in each amplicon that were known to differ between the gene homologues were checked for the presence of the base expected for GH1 and all were confirmed as GH1

Specific areas of the GH-1 gene were amplified as separate ampicons. The location of the amplicons is detailed below in Table 3.

TABLE 3

| Amplicon | | start/txt | end/txt | size |
|---|---|---|---|---|
| 1et | promoter | −1578 | −1229 | 348 |
| 1fu | promoter | −1302 | −928 | 373 |
| 2bq | promoter | −946 | −604 | 341 |
| 2cr | promoter | −670 | −476 | 193 |
| 2ds | promoter | −503 | −225 | 277 |
| 2et | promoter | −278 | 68 | 345 |
| 2fu2 | exon1 | −184 | 127 | 310 |
| 5bq | intron1 | 70 | 392 | 322 |
| 3bq | exon2 | 319 | 591 | 272 |
| 3ds | intron2 | 458 | 767 | 309 |
| 3cr | exon3 | 675 | 893 | 218 |
| 4et | intron3 | 814 | 1034 | 220 |
| 4bq | exon4 | 899 | 1119 | 220 |
| 4fu | intron4 | 1036 | 1391 | 355 |
| 4crl | exon5 | 1292 | 1686 | 394 |
| total | | | | 4497 |

The following primers were used as detailed in Table 4.

TABLE 4

| amplicon | primary primers | secondary primers |
|---|---|---|
| 1et | 1a1/1p1 | 1e1/1t1 |
| 1fu | 1a1/lp1 | 1f1/1u1 |
| 2bq | 2a1/2p1 | 2b1/2q1 |
| 2cr | 2a1/2p1 | 2c1/2r1 |
| 2ds | 2a1/2p1 | 2d1/2s1 |
| 2et | 2a1/2p1 | 2e1/2t1 |
| 2fu | 2a1/2p1 | 2f2/2u1 |
| 3bq | 3a1/3p1 | 3b1/3q1 |
| 3cr | 3a1/3p1 | 3c1/3r1 |
| 3ds | 3a1/3p1 | 3d1/3s1 |
| 4bq | 4a1/4p1 | 4b1/4q1 |
| 4cr | 4a1/4p1 | 4c1/4r1 |
| 4ds | 4a1/4p1 | 4d1/4s1 |
| 4et | 4a1/4p1 | 4e1/4t1 |
| 4fu | 4a1/4p1 | 4f1/4u1 |
| 5bq | 5a1/5p1 | 5b1/5q1 |

The primers referred to are listed below in Table 5.

TABLE 5

| | | |
|---|---|---|
| CRV156.1a1 | tacaggcgtgtgcccaac | SEQ ID NO: 10 |
| CRV156.1e1 | tgccaccacgcccagcta | SEQ ID NO: 11 |
| CRV156.1f1 | atcggaagaaaataatacctcc | SEQ ID NO: 12 |
| GRV156.1p1 | ctgtaatcccagcactttgg | SEQ ID NO: 13 |
| CRV156.1t1 | ctcctcctccttttcagatc | SEQ ID NO: 14 |
| CRV156.1u1 | gatcacgaggtcagtagatc | SEQ ID NO: 15 |
| CRV156.2a1 | ggattcacgccattctcctg | SEQ ID NO: 16 |
| CRV156.2b1 | gtacagagtggatttcacctg | SEQ ID NO: 17 |
| CRV156.2c1 | gtttgtgtctctgctgcaag | SEQ ID NO: 18 |
| CRV156.2d1 | gctgacccaggagtcctc | SEQ ID NO: 19 |
| CRV156.2e1 | ttggccaccatggcctgc | SEQ ID NO: 20 |
| CRV156.2f2 | ccctcacaacactggtgac | SEQ ID NO: 21 |
| CRV156.2p1 | ccccgtcccatctacaggt | SEQ ID NO: 22 |
| CRV156.2q1 | cccctttccctgagcattg | SEQ ID NO: 23 |
| CRV156.2r1 | attgtgggggttgtgagcac | SEQ ID NO: 24 |
| CRV156.2s1 | tgcacagagtgtcagccag | SEQ ID NO: 25 |
| CRV156.2t1 | ttttaggggcgcttacctgt | SEQ ID NO: 26 |
| CRV156.2u1 | cccgtcccatctacaggt | SEQ ID NO: 27 |
| CRV156.3a1 | atttggccaatctcagaaagc | SEQ ID NO: 28 |
| CRV156.3b1 | gctccctctgttgccctc | SEQ ID NO: 29 |
| CRV156.3c1 | ggagctggtctccagcgt | SEQ ID NO: 30 |
| CRV156.3d1 | tatgctccgcgcccatcgt | SEQ ID NO: 31 |
| CRV156.3p1 | atagacgttgctgtcagagg | SEQ ID NO: 32 |
| CRV156.3q1 | ctgcattttcgcttcgggaa | SEQ ID NO: 33 |
| CRV156.3r1 | caggggaaggacgggcat | SEQ ID NO: 34 |
| CRV156.3s1 | gtcggaatagactctgagaaa | SEQ ID NO: 35 |
| CRV156.4a1 | cctccaacagggaggaaaca | SEQ ID NO: 36 |
| CRV156.4b1 | ggcagcacagccaatgcc | SEQ ID NO: 37 |
| CRV156.4c1 | tgagaaagggagggaacagta | SEQ ID NO: 38 |
| CRV156.4d1 | cacacaacgatgacgcacta | SEQ ID NO: 39 |
| CRV156.4e1 | ccaacagggaggaaacacaa | SEQ ID NO: 40 |
| CRV156.4f1 | ctctgacagcaacgtctatg | SEQ ID NO: 41 |
| CRV156.4p1 | tccagcttggttcccaatag | SEQ ID NO: 42 |
| CRV156.4q1 | ctaacacagctctcaaagtca | SEQ ID NO: 43 |
| CRV156.4r1 | cttgccccttgctccatac | SEQ ID NO: 44 |
| CRV156.4s1 | caggttgtcttcccaacttg | SEQ ID NO: 45 |
| CRV156.4t1 | tctaggtcctttaggaggtc | SEQ ID NO: 46 |
| CRV156.4u1 | cgttgtgtgagtttgtgtcg | SEQ ID NO: 47 |
| CRV156.5a1 | gctgacccaggagtcctc | SEQ ID NO: 48 |
| CRV156.5b1 | tcacctagctgcaatggcta | SEQ ID NO: 49 |
| CRV156.5p1 | aaaggccagctggtgcaga | SEQ ID NO: 50 |
| CRV156.5q1 | atggttgggaaggcactgc | SEQ ID NO: 51 |

Primers were diluted to a working stock of 2.5 uM

DNA was diluted to a working stock of 2.5 ng/nl

PCR reactions were carried out in 20 μl. Briefly 4 μl 5X CPCR buffer* was combined with 0.4 μl 10 mM dNTPs, 9.3 μl ddH2O and 0.3 μl PLATINUM™ (Life Technologies Polymerase (5U/μl);

2 μl of each Forward and reverse primer which had been previous diluted to a working stock of 2.5 uM were added along 2 μl of the DNA template previously diluted to 2.5 ng/nl.

* Recipe for 5X CPCR

| | | |
|---|---|---|
| 1.0 | M TrisHCL pH 8.8 | 10.0 ml |
| 4 | M KCL | 1.063 ml |
| 1 | M (NH4)SO4 | 5.0 ml |
| 1 | M MgSO4 | 1.0 ml |
| 20% | Triton | 2.5 ml |

bring volume up to 100 ml.

The following program was used for the primary PCR step in each amplification:

Primary PCR Conditions

5 min at 95° C. initial denaturing DNA;

4 cycles of: 10 sec 96° C. (denaturation), 10 sec 58° C. (annealing), 1.5 min 72° C. (elongation); Followed by 20 cycles of: 10 sec 96° C. (denaturation), 10 sec 55° C. (annealing), 1.5 min 72° C. (elongation) (total of 24 cycles)

After the Primary PCR the product was diluted 1:10 in H2O. The secondary PCR was run according to the following protocol and program.

Secondary PCR Conditions

5 min at 95° C. initial denaturing of DNA;

4 cycles of: 10 sec 96° C. (denaturation), 10 sec 58° C. (annealing), 1.5 min 72° C. (elongation); Followed by 20 cycles of: 10 sec 96° C. (denaturation), 10 sec 55° C. (annealing), up to 1 min 72° C. (elongation) (total of 24 cycles)

Amplicon DNA was obtained from each patient sample and sequenced.

Sequencing Protocol

Primers for the secondary PCR are tailed with M13 sequences. PCR products from the secondary PCR are diluted 1:10 in 1 mM EDTA and submitted for sequencing reactions using dye-primer chemistry and sequencing primers complementary to the M13 tails. Sequencing products were run on capillary sequencers (MegaBace, Molecular Dynamics) or ABI377 sequencers. Raw traces were analyzed base-called using proprietary software.

Results

As a result of following the above protocol and the protocol of Example 1, the following coding region mutations were found. The reference to “position” refers to the numbering system of FIG. 1.

TABLE 6

| Position | Location | Base | AA Change | Site | Bronson | PPGx | CRV | GCI (182) |
|---|---|---|---|---|---|---|---|---|
| 69 | Exon 1 | A/G | Thr- | Signal | 1 | 6 | 6 | 5 |
| 377* | Exon 2 | C/T | Pro-8/Ser | Signal | | 1 | | |
| 438** | Exon 2 | C/T | Ala13/Val | Near S 1 1 | | | 1 | 1 |
| 473 | Exon 2 | T/A | Phe25/Ile | Site 1 | | | | 1 |
| 474 | Exon 2 | T/A | Phe25/Tyr | Site 1 | | 1 | | 1 |
| 485 | Exon 2 | C/T | Gln29/TER | Site 1 | | | 1 | |
| 748 | Exon 3 | A/G | Asn47/Asp | Site 1 | | | | |
| 749 | Exon 3 | AA/CC | Asn47/Thr | Site 1 | | 1 | | |
| 749 | Exon 3 | A/C | Asn47/Thr | Site 1 | | 1 | | |
| 937 | Exon 4 | C/G | Ser79/Cys | Helix 2 | 1 | | | |
| 1411 | Exon 5 | G/C | Asp153/His | Loop | 1 | | | |

*short individual in Michigan population
*proline to serine change in leader sequence can affect folding and function
**change from Ala13 to Val involves the contact area between helices 1 ad 3 and site 2 binding to the receptor

It should be noted that coding mutations within the Site 1 binding region are liable to be strongly associated with function. Although Ala 13 is technically outside of the binding area it is part of the hydrophobic core of helix 1 interacting with helix 3 and 4. Although it is buried, a mutation to valine may interfere with site 2 binding, since it is positioned close to this site. A substitution valine may cause a destabilization of helix 1 in the site 2 binding region.”

IGF1 and and its binding protein, IGF1-BP3, are normally upregulated by GH1 and promote many of the growth effects of GH1. We have measured the IGF1 and IGF1-BP3 plasma levels from the subjects in the GCI cohort. The plasma levels of IGF1 with age, but for all ages a value below 100 ng/ml is considered low. Except for one individual carrying multiple, possibly compensating mutations, the IGF1 values of the GCI subjects carrying coding changes in their GH1 gene are below the normal level. IGF1-BP3 values below 3 mg/l are considered low. Most of the subjects, except one carrying a mutation at position 69, have low IGF-BP3 values.

That data is presented below in Table 7

TABLE 7

| Position | Subject | IGF-1 (ng/ml) | IGF1-BP3 (mg/lt) |
|---|---|---|---|
| 69 | QU6G3 | 55 | 3.9 |
| 69 | NVNJV | 85 | 2.4 |
| 69 | QUQLM | 73 | 2.2 |
| 69 | NSM16 | 69 | 1.1 |
| 69 | VJ4KRD | 165 | 2.2 |
| 438 | GEGZ8 | 82 | 1.6 |
| 473 | 1ER1Q | 80 | 2.1 |
| 474 | VJ4KRD | 165 | 2.2 |

Association Studies

Once a polymorphism is identified, as noted above, it becomes desirable to determine which form(s) of an identified polymorphism are present in individuals under test for diagnostic and predictive purposes or for establishing a correlation between other phenotypes and the presence of a particular polymorphism.

In determining the identity of a particular nucleotide position there are a variety of suitable procedures, which are discussed in turn.

Analysis of Polymorphisms

A. Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

Many of the methods described below require amplification of DNA from target samples. This can be accomplished by PCR. See generally PCR Technology: Principles and Applications for DNA Amplification (ed. H. A. Erlich, Freeman Press, N.Y., N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Detection of Polymorphisms in Target DNA

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

These probes are characterized in that they preferably comprise between 8 and 50 nucleotides, and in that they are sufficiently complementary to a sequence comprising a polymorphic marker of the present invention to hybridize thereto and preferably sufficiently specific to be able to discriminate the targeted sequence for only one nucleotide variation. The GC content in the probes of the invention usually ranges between 10 and 75%, preferably between 35 and 60%, and more preferably between 40 and 55%. The length of these probes can range from 10, 15, 20, or 30 to at least 100 nucleotides, preferably from 10 to 50, more preferably from 18 to 35 nucleotides. A particularly preferred probe is 25 nucleotides; in length. Preferably the polymorphic marker is within 4 nucleotides of the center of the polynucleotide probe. In particularly preferred probes the polymorphic marker is at the center of said polynucleotide. Shorter probes may lack specificity for a target nucleic acid sequence and generally require cooler temperatures to form sufficiently stable hybrid complexes. with the template. Longer probes are expensive to produce and can sometimes self-hybridize to form hairpin structures. Methods for the synthesis of oligonucleotide probes have been described above and can be applied to the probes of the present invention.

Preferably the probes of the present invention are labeled or immobilized on a solid support. Labels and solid supports are well known in the art. Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′hydroxyl group simply can be cleaved, replaced or modified,

The probes of the present invention are useful for a number of purposes. They can be used in Southern hybridization to genomic DNA or Northern hybridization to mRNA. The probes can also be used to detect PCR amplification products. By assaying the hybridization to an allele. specific probe, one can detect the presence or absence of a biallelic marker allele in a given sample.

High-Throughput parallel hybridizations in array format are specifically encompassed within “hybridization assays” and are described below.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Allele-Specific Primers An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer, which hybridizes at a distal site. Amplification proceeds from the two primers leading to a detectable product signifyng the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3'-most position of the oligonucleotide aligned with the polymorphism because this position is most destabilizing to elongation from the primer. See, e.g., WO 93/22456. The invention of course, contemplates such primers with distal mismatches as well as primers, which because of chosen conditions form unstable base pairing and thus prime inefficiently.

3. Direct-Sequencing

The direct analysis of the sequence of polymorphisms of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988). It should be recognized that the field of DNA sequencing has advanced considerably in the past several years and that the invention contemplates such advances. Most notably, within the past decade there has been increasing reliance on automated DNA sequence analysis.

4. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

5. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures, which are partially dependent on the base sequence. The different electrophoretic mobilities of single-stranded amplification products can be related to base-sequence difference between alleles of target sequences.

Other modifications of the methods above exist, including allele-specific hybridization on filters, allele-specific PCR, PCR plus restriction enzyme digest (RFLP-PCR), denaturing capillary electrophoresis, primer extension and time-of-flight mass spectrometry, and the 5′ nuclease (Taq-Man™) assay.

The Taq-Man assay takes advantage of the 5′ nuclease activity of Taq DNA polymerase to digest a DNA probe annealed specifically to the accumulating amplification product. Taq-Man probes are labeled with a donor-acceptor dye pair that interacts via fluorescence energy transfer. Cleavage of the Taq-Man probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., Nature Genetics, 9:341-342, 1995). In an alternative homogeneous hybridization-based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., Nature Biotechnology, 16:49-531 1998).

Preferred techniques for SNP genotyping should allow large scale, automated analysis which do not require extensive optimization for each SNP analyzed. Examples of the later are DASH (Dynamic Allele-Specific hybridization) which is amenable to formatting in microtiter plates (Hybaid) and “single-stringency” DNA-chip hybridization (Affymetrix)” It should be recognized of course, that this list is not inclusive.

High-Throughput parallel hybridizations in array format are specifically encompassed by the invention and are described below.

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA I gene, in S. cerevisiae mutant strains, and in the protease gene of HIV-I virus (Hacia et al., Nature Genetics, 14(4):441-447, 1996; Shoemaker et al., Nature Genetics, 14(4):450-456, 1996 Kozal et al., Nature Medicine, 2:753-759, 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP785280 describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e. nucleotides. Tiling strategies are further described in PCT application No. WO 95/11995. In a particular aspect, arrays are tiled for a number of specific, identified biallelic marker sequences. In particular the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual crosshybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of an isolated polynucleotide comprising between 6-800 contiguous nucleotides of SEQ ID No. 1 and the sequences complementary thereto, or a fragment thereof at least about 8 consecutive nucleotides, preferably 10, 15, 20, more preferably 25, 30, 40, 47, or 50 consecutive nucleotides, including at least one polymorphic site. In some embodiments, the chip may comprise an array of at least 2, 3, 4, 5, 6, 7, 8 or more of these polynucleotides of the invention. Solid supports and polynucleotides of the present invention attached to solid supports are further described in 1.

Fluorescent Allele-Specific PCR (FAS-PCR) uses allele specific primers which differ by a single 3′ nucleotide which is an exact match to the allele to be detected (Howard et al. 1999). Thus, two primers designed to match exactly each allele of a biallelic SNP are used with a single, common, reverse primer to detect each of the allele specific primers. This uses to advantage the observation that if the 3′ nucleotide of the PCR amplification primer does not match exactly, then amplification will not be successful. Typically, each allele specific primer is tagged with a different fluorescent primer to allow their discrimination when analyzed by gel or capillary electrophoresis using an automated DNA Analysis System such as the PE Biosystems Models 310/373/377 or 3700.

SNPs also can be genotyped rapidly and efficiently using techniques that make use of thermal denaturation differences due to differences in DNA base composition. In one embodiment of this test, allele specific primers are designed as above to detect biallelic SNP with the exception that to one primer is added a 5′ GC tail of 26 bases (Germer and Higuichi, 1999). After PCR amplification with a single, common reverse primer, a fluorescent dye that binds preferentially to dsDNA (e.g., SYBR Green 1) is added to the tube and then the thermal denaturation profile of the dsDNA product of PCR amplification is determined. Samples homozygous for the SNP amplified by the GC tailed primer will denature at the high end of the temperature scale, while samples homozygous for the SN amplified by the non-GC tagged primer will denature at the low end of the temperature scale. Heterozygous samples will show two peaks in the thermal denaturation profile.

In a variant of the foregoing technique, dynamic allele-specific hybridization (DASH) is detected by thermal denaturation curves (Howell et al., 1999). In on embodiment of this test, a pair of PCR primers is used to amplify the genomic region in the DNA sample containing the SNP. One of these primers is biotinylated to allow subsequent binding of the biotinylated product strand to strepavidin-coated microtiter plates while the non-biotinylated strand is washed away with alkali. An oligoucleotide probe which is an exact match for one allele is hybridized to the immobilized PCR product at low temperature. This forms a dsDNA region that interacts with a dsDNA intercalating dye (e.g., SYBR Green 1). The thermal denaturation profile then allows the test to distinguish the single base mismatch between the biallelic SNP due to the difference in melting temperature. Other methods for SNP genotyping and their application to the detection of SNP in the GH-1 gene can be envisaged by one skilled in the art.

Polymorphisms of the Invention in Methods of Genetic Diagnostics

The polymorphisms of the present invention can also be used to develop diagnostics tests capable of identifying individuals who are at increased risk of developing GH-1 dysfunction or who suffer from GH-1 dysfunction. The diagnostic techniques of the present invention may employ a variety of methodologies to determine whether a test subject has a polymorphic marker pattern associated with an increased risk of developing GH-1 dysfunction or whether the individual suffers from GH-1 dysfunction coincident with carrying a particular mutation, including methods which enable the analysis of individual chromosomes for haplotyping, such as family studies, single sperm DNA analysis or somatic hybrids as well as antibody based methods designed to detect the polymorphisms at the protein level.

Determining the Haplotype of an Individual

It is often particularly advantageous to determine the identity of nucleotides occupying specific polymorphic sites on the same chromosomal segment in an individual (the haplotype). The present invention therefore further provides a method of diagnosing a GH-1 dysfunction, or the propensity of an individual to transmit GH-1 dysfunction to offspring, or determining a predisposition to GH-1 dysfunction by determining the presence or absence of a GH-1 haplotype in a patient by obtaining material comprising nucleic acid including the GH-1 polymorphic sites from the patient; enzymatically amplifying the nucleic acid using pairs of oligonucleotide primers complementary to nucleotide sequences flanking any of the polymorphic sites at position, within SEQ ID NO:1 or 4 to produce amplified products containing any of the polymorphic site or other GH-1 polymorphic sites and determining the GH-1 haplotype.

In order to determine a haplotype one skilled in the art understands that an amplified product can be sequenced directly or subcloned into a vector prior to sequence analysis. Commercially available sequencing kits including the Sequenase TM kit from Amersham Life Science (Arlington Heights, Ill.) can be used to sequence an amplified product in the methods of the invention. Automated sequence analysis also can be useful, and automated sequencing instruments such as the Prism 377 DNA Sequencer or the 373 DNA Sequencer are commercially available, for example, from Applied Biosystems (Foster City, Calif.; see, also, Frazier et al., Electrophoresis 17:1550-1552 (1996), which is incorporated herein by reference). Both copies in a diploid genome give rise to sequence the haplotypic composition of an individual can thus be inferred from direct sequence analysis.

Another possibility is that single chromosomes can be studied independently, for example, by asymmetric PCR amplification (see Newton et al., Nucleic Acids Res., 17:2503-2516, 1989; Wu et al., Proc. Natl Acad Sci. USA, 86:2757, 1989) or by isolation of single chromosome by limit dilution followed by PCR amplification (see Ruano et al., Proc. Natl Acad. Sci. USA, 87:6296-6300, 1990). Further, a sample may be haplotyped for sufficiently close polymorphic markers by double PCR amplification of specific alleles (Sarkar, G. and Sommer S. S., Biotechniques, 1991).

The present invention provides diagnostic methods to determine whether an individual is at risk of developing GH-1 dysfunction or suffers from GH-1 dysfunction coincident with a mutation or a polymorphism in of the present invention. The present invention also provides methods to determine whether an individual is likely to respond positively to an agent acting on GH-1 dysfunction disorder or whether an individual is at risk of developing an adverse side effect to an agent acting on GH-1 dysfunction

These methods involve obtaining a nucleic acid sample from the individual and, determining, whether the nucleic acid sample contains at least one allele or at least one polymorphic haplotype, indicative of a risk of developing the trait or indicative that the individual expresses the trait as a result of possessing trait-causing allele.

Preferably, in such diagnostic methods, a nucleic acid sample is obtained from the individual and this sample is genotyped using methods described above. The diagnostics may be based on a single polymorphism or on a group of polymorphisms. In each of these methods, a nucleic acid sample is obtained from the test subject and the polymorphic pattern of one or more of the polymorphic markers listed in Table 1.

One would conclude therefore that an individual suffers from GH-1 dysfunction and/or may be in need of treatment with an agent acting on GH-1 dysfunction if one or more of the following conditions exist:

(a) the identity of the nucleotide at S1 on the coding strand is C or G on the non-coding strand

(b) the identity of the nucleotide at S2 on the coding strand is T or A on the non-coding strand

(c) the identity of the nucleotide at S3 on the coding strand is T or A on the non-coding strand

(d) the identity of the nucleotide at S4 on the coding strand is A or T on the non-coding strand

(e) the identity of the nucleotide at S5 on the coding strand is A or T on the non-coding strand

(f) the identity of the nucleotide at S6 on the coding strand is T or A on the non-coding strand

(g) the identity of the nucleotide at S7 on the coding strand is C or G on the non-coding strand

(h) the identity of the nucleotide at S8 on the coding strand is G or C on the non-coding strand

(i) the identity of the nucleotide at S9 on the coding strand is C or G on the non-coding strand.

In one embodiment, PCR amplification is conducted on the nucleic acid sample to amplify regions in which polymorphisms associated with a detectable phenotype have been identified. The amplification products are sequenced to determine whether the individual possesses one or more polymorphisms associated with a detectable phenotype. The primers used to generate amplification products may comprise the primers listed in Examples 1 and 2. Alternatively, the nucleic acid sample is subjected to microsequencing reactions as described above to determine whether the individual possesses one or more polymorphisms associated with a detectable phenotype resulting from a mutation or a polymorphism. in a candidate gene. The primers used in the microsequencing reactions may include the primers listed in Examples 1 and 2. In another embodiment, the nucleic acid sample is contacted with one or more allele specific oligonucleotide probes which, specifically hybridize to one or more candidate gene alleles associated with a detectable phenotype.

In a preferred embodiment the identity of the nucleotide present at, at least one, biallelic marker selected from the group consisting the polymorphic sites at position, the nucleotides at position, 68, 116, 177, 212, 213, 224, 279, 375 or 596 of SEQ ID NO:1 or positions 1665, 1973, 2034, 2069, 2070, 2081, 2345, 2533 or 3007 of SEQ ID NO:4, is determined and the detectable trait is GH-1 dysfunction.

These diagnostic methods are extremely convenient both for the patient and the clinician. The test sample obtained from the patient in the detection method of the invention preferably comprises genomic DNA extracted from patient lymphocytes by standard procedures, such as from buccal smears, blood samples or hair. GH-1 gene analysis is thereafter carried out by any suitable for identifying a nucleotide at a particular position within the GH-1 gene. Diagnostic kits comprising polynucleotides of the present invention are further described below.

Antibodies of the Invention

We note that all of the SNPs in the coding region which change an amino acid would be amenable to antibody-based diagnostics.

Polyclonal and/or monoclonal antibodies that specifically bind to variant gene products but not to corresponding reference gene products are contemplated. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, N.Y. (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition. Diagnostics using such antibodies are well known in the art and can include but are not limited to Western Blot analysis, ELISA analysis and radioimmunoassay.

Once polyclonal and/or monoclonal antibodies that specifically bind to variant gene products but not to corresponding reference gene products are in hand a host of diagnostics are within the reach of one of ordinary skill in the art. Such antibodies also have utility as therapeutic modalities.

It is contemplated that same panoply of predictive methods for diagnosing GH-1 dysfunction on a nucleic acid level could be specific antibodies.

Diagnostic Kits

The invention further provides kits comprising at least one allele-specific oligonucleotide or antibody as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting both of the polymorphisms described. Optional additional components of the kit include, for example, restriction enzymes, reverse-transcriptase or polymerase, the substrate nucleoside triphosphates, means used to label (for example, an avidinenzyme conjugate and enzyme substrate and chromogen if the label is biotin), and the appropriate buffers for reverse transcription, PCR, or hybridization reactions. Usually, the kit also contains instructions for carrying out the methods.

The present invention is used to determine whether or not an individual has an GH-1 polymorphism which has been associated with GH-1 dysfunction. Such GH-1 polymorphisms are shown to be genetic risk factors in population studies which compare the frequency of the said polymorphism in the general population and the frequency of the polymorphism in persons with GH-1 dysfunction. If for example, said polymorphism occurs at a frequency of 3% in the general population, but at a frequency of 30% in persons with GH-1 dysfunction, then a test for said polymorphism will reveal individuals having a higher likelihood of having or developing a GH-1 dysfunction related disorder. This information may be used either prognostically to identify individuals with increased risk for developing GH-1 dysfunction at a future point in time, or diagnostically to identify individuals presenting with GH-1 dysfunction on clinical exam who may therefore be diagnosed as being more likely to have GH-1 dysfunction related disorder.

Analysis of said GH-1 polymorphism for the purpose of prognosis or diagnosis may be performed by one of any techniques capable of accurately detecting SNP including but not limited to allele-specific hybridization on filters, allele-specific PCR, PCR plus restriction enzyme digest (RFLP-PCR), denaturing capillary electrophoresis, primer extension and time-of-flight mass spectrometry, and the 5′ nuclease (Taq-Man) assay.

Preferred techniques for SNP genotyping should allow large scale, automated analysis which do not require extensive optimization for each SNP analyzed. Examples of the later are DASH (Dynamic Allele-Specific hybridization) which is amenable to formatting in microtiter plates (Hybaid) and “single-stringency” DNA-chip hybridization (Affymetrix).

Polypeptides and Encoding Nucleic Acid of the Invention

The invention comprises GH-1 mutant polypeptides (and encoding nucleic acids) which are a GH-1 polypeptides encoded by GH-1 gene or transcript or a portion thereof which comprises at least one GH-1 polymorphic site with the polymorphic site encoding the rare allele as shown in Table 1. Therefore, the term GH-1 mutant polypeptide encompasses a polypeptide species comprising SEQ ID NO:3 wherein one or more of positions 13, 25, 29, 47, 79 or 153 is occupied by the amino acid coded for by the rare allele. (i.e. position 13=Val, position 25=Ile or Tyr, position 47=Thr, position 79=Cys, and/or position 153=His or conservative substitutions at these positions). It will be appreciated that the numbering system here makes reference to the numbering relative to the most abundant isoform of the GH-1 protein. The invention also comprises unprocessed GH-1 mutant polypeptides having a leader or signal sequence attached and would specifically encompass unprocessed GH-1 mutant polypeptides having polymorphic substitutions in the signal or leader sequence as well.

Such mutant proteins have utility as antagonists of GH-1 hormone action. Mutant proteins with mutations effecting site 2 binding are particularly preferred. It is specifically contemplated that polynucleotides encoding the GH-1 mutant polypeptides are useful agents of gene therapy and such polynucleotides encoding the mutant proteins are part of the invention. It is appreciated that the invention also comprises polynucleotides encoding the GH-1 mutant proteins as exemplified by SEQ ID NO:1 and SEQ ID NO:4 and any alternative splice products of the GH-1 locus.

As is well known in the art, due to the degeneracy of the genetic code, there are numerous other DNA and RNA molecules that can code for the same polypeptide as that encoded by the aforementioned mutant GH-1 mutant polypeptides. The present invention, therefore, contemplates those other DNA and RNA molecules which, on expression, encode the polypeptides.

Methods of Genetic Analysis Using the Polymorphic Markers of the Present Invention

Once the identity of a polymorphism has been established it becomes desirable to attempt to associate a particular form of the polymorphism with the presence or absence of a phenotype other than growth hormone dysfunction.

It is apparent that while we have established an association of certain polymorphisms of the invention with a GH-1 dysfunction phenotype, the invention also contemplates the use of the polymorphic sites of the invention as markers for the analysis of other disease states, of susceptibility to drug treatment for GH-1 dysfunction or other diseases, or may be included in any complete or partial genetic map of the human genome.

The polymorphic markers of the present invention find use in any method known in the art to demonstrate a statistically significant correlation between a genotype and a phenotype. Different methods are available for the genetic analysis of complex traits (see Lander and Schork, Science, 265, 2037-2048, 1994). To determine if a polymorphism is associated with a phenotypic trait three main methods are used: the linkage approach (either parametric or non-parametric) in which evidence is sought for cosegregation between a locus and a putative trait locus using family studies, and the association approach in which evidence is sought for a statistically significant association between an allele and a trait or a trait causing allele and the TDT approach which tests for both linkage and association.

The polymorphic markers may be used in parametric and non-parametric linkage analysis methods. Preferably, the polymorphic markers of the present invention are used to identify genes associated with GH-1 dysfunction or other disorders using association studies such as the case control method, an approach which does not require the use of affected families and which permits the identification of genes associated with complex and sporadic traits.

The genetic analysis using the polymorphic markers of the present invention may be conducted on any scale. The whole set of polymorphic markers of the present invention or any subset of polymorphic markers of the present invention may be used. Further, any set of genetic markers including a polymorphic marker of the present invention may be used. A set of biallelic polymorphisms that, could be used as genetic markers in combination with the polymorphic markers of the present invention, has been described in WO 98/20165. As mentioned above, it should be noted that the polymorphic markers of the present invention may be included in any complete or partial genetic map of the human genome. These different uses are specifically contemplated in the present invention.

It will be clear that the invention may be practiced otherwise than as particularly described in the foregoing description and examples.

Numerous modifications and variations of the present invention are possible in light of the above teachings and, therefore, are within the scope of the invention

The entire disclosures of all publications cited herein are hereby incorporated by reference.

What is claimed is:

1. An isolated GH-1 diagnostic polynucleotide or its complement comprising between 10 and 800 contiguous nucleotides.

2. The isolated GH-1 diagnostic polynucleotide of claim 1 which is derived from genomic DNA.

3. The isolated GH-1 diagnostic polynucleotide of claim 2 which is derived from the sequence delineated in SEQ ID NO:4

4. The isolated GH-1 diagnostic polynucleotide of claim which is derived from messenger RNA.

5. The isolated polynucleotide of claim 1 in which the polymorphic site is S1 and the nucleotide at the polymorphic site is selected from the group of nucleotides A or C

6. The isolated polynucleotide of claim 1 in which the polymorphic site is S2 and the nucleotide at the polymorphic site is selected from the group of nucleotides C or T

7. The isolated polynucleotide of claim 1 in which the polymorphic site is S3 and the nucleotide at the polymorphic site is selected from the group of nucleotides C or T.

8. The isolated polynucleotide of claim 1 in which the polymorphic site is S4 and the nucleotide at the polymorphic site is selected from the group of nucleotides T or A.

9. The isolated polynucleotide of claim 1 in which the polymorphic site is S5 and the nucleotide at the polymorphic site is selected from the group of nucleotides T or A.

10. The isolated polynucleotide of claim 1 in which the polymorphic site is S6 and the nucleotide at the polymorphic site is selected from group of nucleotides C or T

11. The isolated polynucleotide of claim 1 in which the polymorphic site is S7 and the nucleotide at the polymorphic site is selected from the group of nucleotides A or C.

12. The isolated polynucleotide of claim 1 in which the polymorphic site is S8 and the nucleotide at the polymorphic site is selected from the group of nucleotides C or G.

13. The isolated polynucleotide of claim 1 in which the polymorphic site is S9 and the nucleotide at the polymorphic site is selected from group of nucleotides C or G.

14. The isolated polynucleotide of claim 1 that is less than 400 nucleotides

15. The isolated polynucleotide of claim 1 that is less than 50 nucleotides.

16. The isolated polynucleotide of claim 1 that is less than 30 nucleotides.

17. The isolated polynucleotide of claim 1 that is less than 25 nucleotides

18. The isolated polynucleotide of claim 1 wherein the polymorphism is within 4 nucleotides of the center of said polynucleotide.

19. The isolated polynucleotide of claim 1 wherein the polymorphism is at the center of said polynucleotide.

20. The isolated polynucleotide of claim 1 wherein the polymorphism is at the end of said polynucleotide.

21. The isolated polynucleotide of claim 1 wherein the polynucleotide is a probe.

22. The isolated polynucleotide of claim 1 wherein the polynucleotide is a primer.

23. A polynucleotide for use in amplifying a segment of SEQ ID NO:4 comprising a polymorphic site.

24. A single-stranded DNA probe that hybridizes to a variant GH-1 gene and not to a wild type GH-1 gene, wherein the variant GH-1 gene is selected from the group consisting of:
SEQ ID NO:4 having a “C” at position 1665,
SEQ ID NO:4 having a “T” at position 1973,
SEQ ID NO:4 having a “T” at position 2034,
SEQ ID NO:4 having a “A” at position 2069,
SEQ ID NO:4 having a “A” at position 2070,
SEQ ID NO:4 having a “T” at position 2081,
SEQ ID NO:4 having a “C” at position 2345
SEQ ID NO:4 having a “G” at position 2533
SEQ ID NO:4 having a “G” at position 3007

25. An array of nucleic acid molecules attached to a solid support, the array comprising a single stranded DNA probe according to claim 24.

26. A method for classifying a nucleic acid molecule encoding GH-1 or a fragment thereof obtained from an individual for diagnostic or prognostic purposes, comprising;
determining the identity of a nucleotide from said nucleic acid which corresponds to the nucleotide occupying at least one GH-1 polymorphic site selected from the group consisting of: S1, S2, S3, S4, S5, S6, S7, S8 and S9 on either the coding or non-coding strand.

27. The method of claim 26, wherein the determining comprises determining the identity of the nucleotide of at least two GH-1 polymorphic sites.

28. A method of evaluating therapy with an agent acting on GH-1 dysfunction for treatment of a patient, comprising:
(a) determining the identity of a nucleotide from a nucleic acid obtained from said patient which corresponds to the nucleotide occupying at least one GH-1 polymorphic site on either the coding or non-coding strand;
(b) evaluating whether said patient should undergo therapy with said agent.

29. The method of claim 28 wherein the evaluating comprises:
determining that the patient should undergo therapy with said agent if any of the following conditions exist:
(a) the identity of the nucleotide at S1 on the coding strand is C or G on the non-coding strand
(b) the identity of the nucleotide at S2 on the coding strand is T or A on the non-coding strand
(c) the identity of the nucleotide at S3 on the coding strand is T or A on the non-coding strand
(d) the identity of the nucleotide at S4 on the coding strand is A or T on the non-coding strand
(e) the identity of the nucleotide at S5 on the coding strand is A or T on the non-coding strand
(f) the identity of the nucleotide at S6 on the coding strand is T or A on the non-coding strand
(g) the identity of the nucleotide at S7 on the coding strand is C or G on the non-coding strand
(h) the identity of the nucleotide at S8 on the coding strand is G or C on the non-coding strand
(i) the identity of the nucleotide at S9 on the coding strand is C or G on the non-coding strand.

30. The method of claim 28 wherein said agent is human growth hormone.

31. A method of administering human growth hormone comprising administering human growth hormone to a patient previously determined to have a nucleotide at a GH-1 polymorphic site indicating GH-1 dysfunction wherein the previous determination has ascertained that any of the following conditions exist:
(a) the identity of the nucleotide at S1 on the coding strand is C or G on the non-coding strand
(b) the identity of the nucleotide at S2 on the coding strand is T or A on the non-coding strand
(c) the identity of the nucleotide at S3 on the coding strand is T or A on the non-coding strand
(d) the identity of the nucleotide at S4 on the coding strand is A or T on the non-coding strand
(e) the identity of the nucleotide at S5 on the coding strand is A or T on the non-coding strand
(f) the identity of the nucleotide at S6 on the coding strand is T or A on the non-coding strand
(g) the identity of the nucleotide at S7 on the coding strand is C or G on the non-coding strand
(h) the identity of the nucleotide at S8 on the coding strand is G or C on the non-coding strand
(i) the identity of the nucleotide at S9 on the coding strand is C or G on the non-coding strand.

32. A method of selecting a therapy for a patient comprising,
(a) determining the identity of a nucleotide which corresponds to the nucleotide occupying at least one GH-1 polymorphic site selected from the group consisting of: S1, S2, S3, S4, S5, S6, S7, S8 and S9. on either the coding or non-coding strand;
(b) transmitting a descriptor of therapy selected based on the identity of the nucleotide at said GH-1 polymorphic site.

33. A method of haplotype determination in an individual for diagnostic or prognostic purposes, comprising
determining a nucleotide on a single chromosome. which corresponds to the nucleotide occupying one or more GH-1 polymorphic sites selected from the group consisting of: S1, S2, S3, S4, S5, S6, S7, S8 and S9.

34. A diagnostic kit comprising the required components for the determination of the of the identity of the nucleotide or nucleotides occupying a GH-1 polymorphic site selected from the group consisting of: S1, S2, S3, S4, S5, S6, S7, S8 and S9 in small volumes in a self contained kit.

35. The diagnostic kit of claim 34 comprising an isolated GH-1 diagnostic polynucleotide comprising between 10 and 800 contiguous nucleotides.

36. An antibody selected from the group of antibodies consisting of:
(a) an antibody to an epitope comprising amino acid position 3 of SEQ ID NO:2 capable of distinguishing a threonine from an alanine at that amino acid position; or
(b) an antibody to an epitope comprising amino acid position 19 of SEQ ID NO: 2 capable of distinguishing a proline from a serine at that amino acid position;
(c) an antibody to an epitope comprising amino acid position 13 of SEQ ID NO: 3 capable of distinguishing an alanine from a valine at that amino acid position;
(d) an antibody to an epitope comprising amino acid position 25 of SEQ ID NO: 3 capable of distinguishing phenylalanine from isoleucine or tyrosine at that amino acid position;
(e) an antibody to an epitope comprising amino acid position 28 of SEQ ID NO: 3 capable of identifying a terminal tyrosine at that amino acid position;
(f) an antibody to an epitope comprising amino acid position 47 of SEQ ID NO: 3 capable of distinguishing an asparagine from threonine at that amino acid position;
(g) an antibody to an epitope comprising amino acid position 79 of SEQ ID NO:3 capable of distinguishing a serine from a cysteine at that amino acid position.
(h) an antibody to an epitope comprising amino acid position 153 of SEQ ID NO:3 capable of distinguishing an aspartic acid from histidine at that amino acid position.

37. A diagnostic kit comprising the antibody of claim 36.

38. A isolated GH-1 mutant polypeptide comprising one or more of the following mutations:
(a) the amino acid encoded by the GH-1 polymorphic site S3 is a valine
(b) the amino acid encoded by the GH-1 polymorphic site S4 is a isoleucine
(c) the amino acid encoded by the GH-1 polymorphic site S5 is a tyrosine
(d) the amino acid encoded by the GH-1 polymorphic site S7 is a threonine
(e) the amino acid encoded by the GH-1 polymorphic site S8 is a cysteine
(f) the amino acid encoded by the GH-1 polymorphic site S9 is a histidine

39. The isolated mutant polypeptide of claim 38 which comprises one mutation.

40. An isolated polynucleotide encoding the GH-1 mutant polypeptide of claim 38.

41. A method for treating a disease state comprising the step of administering to a patient in need of such treatment an amount of a GH-1 mutant polypeptide sufficient to alter GH-1 activity in the tissues of said patient.

42. A method for classifying a GH-1 polypeptide obtained from an individual for diagnostic or prognostic purposes, to determine whether said polypeptide is a GH-1 mutant polypeptide comprising;
determining the identity of an amino acid encoded by at least one GH-1 polymorphic site selected from the group consisting of: S1, S2, S3, S4, S5, S6, S7, S8 and S9.

43. The method of claim 42, wherein the determining comprises determining the identity of an amino acid encoded by at least two GH-1 polymorphic sites.

44. A method of evaluating therapy with an agent acting on GH-1 dysfunction for treatment of a patient, comprising:
(a) determining whether a GH-i polypeptide obtained from said patient is a GH-1 mutant polypeptide;
(b) evaluating whether the patient should undergo therapy with said agent.

45. The method of claim 44 wherein the evaluating comprises: determining that the patient should undergo therapy with said agent if any of the following conditions exist:
(a) the identity of the amino acid encoded by the GH-1 polymorphic site S1 is an alanine
(b) the identity of the amino acid encoded by the GH-1 polymorphic site S2 is a serine
(c) the identity of the amino acid encoded by the GH-1 polymorphic site S3 is a valine
(d) the identity of the amino acid encoded by the GH-1 polymorphic site S4 is a isoleucine
(e) the identity of the amino acid encoded by the GH-1 polymorphic site S5 is a tyrosine
(f) the identity of the amino acid adjacent to the the GH-1 polymorphic site S6 is a terminal tyrosine
(g) the identity of the amino acid encoded by the GH-1 polymorphic site S7 is a threonine
(h) the identity of the amino acid encoded by the GH-1 polymorphic site S8 is a cysteine
(i) the identity of the amino acid encoded by the GH-1 polymorphic site S9 is a histidine.

46. The method of claim 44 wherein said agent is human growth hormone.

47. A method of administering human growth hormone comprising administering human growth hormone to a patient previously determined to express a mutant GH-1 polypeptide wherein the previous determination has ascertained that any of the following conditions exist:
(a) the identity of the amino acid encoded by the GH-1 polymorphic site S1 is an alanine
(b) the identity of the amino acid encoded by the GH-1 polymorphic site S2 is a serine
(c) the identity of the amino acid encoded by the GH-1 polymorphic site S3 is a valine
(d) the identity of the amino acid encoded by the GH-1 polymorphic site S4 is a isoleucine
(e) the identity of the amino acid encoded by the GH-1 polymorphic site S5 is a tyrosine
(f) the identity of the amino acid adjacent to the the GH-1 polymorphic site S6 is a terminal tyrosine
(g) the identity of the amino acid encoded by the GH-1 polymorphic site S7 is a threonine
(h) the identity of the amino acid encoded by the GH-1 polymorphic site S8 is a cysteine
(i) the identity of the amino acid encoded by the GH-1 polymorphic site S9 is a histidine

48. A method of selecting a therapy for a patient comprising,
(a) determining whether a GH-1 polypeptide obtained from said patient is a GH-1 mutant polypeptide
(b) transmitting a descriptor of therapy selected based on the identity of an amino acid encoded by a GH-1 polymorphic site.
